SEQUENCE LISTING (47 sequences)

SEQ ID NO: 1 - protein, 458 aa, Homo sapiens
  SOURCE 1..458: /mol_type=“protein” /organism=“Homo sapiens”
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 434 aa, Homo sapiens
  SOURCE 1..434: /mol_type=“protein” /organism=“Homo sapiens”
GKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 3 - protein, 47 aa, Homo sapiens
  SOURCE 1..47: /mol_type=“protein” /organism=“Homo sapiens”
IGLGIFFCVRCRHRRRQAERMSQIKRLLSEKKTCQCPHRFQKTCSPI

SEQ ID NO: 4 - protein, 36 aa, Homo sapiens
  SOURCE 1..36: /mol_type=“protein” /organism=“Homo sapiens”
GKKVVLGKKGDTVELTCTASQKKSIQFHWKNSNQIK

SEQ ID NO: 5 - protein, 15 aa, Homo sapiens
  SOURCE 1..15: /mol_type=“protein” /organism=“Homo sapiens”
DTYICEVEDQKEEVQ

SEQ ID NO: 6 - protein, 25 aa, Homo sapiens
  SOURCE 1..25: /mol_type=“protein” /organism=“Homo sapiens”
KEEVQLLVFGLTANSDTHLLQGQSL

SEQ ID NO: 7 - protein, 166 aa, Homo sapiens
  SOURCE 1..166: /mol_type=“protein” /organism=“Homo sapiens”
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 8 - protein, 166 aa, Homo sapiens
  SOURCE 1..166: /mol_type=“protein” /organism=“Homo sapiens”
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 9 - protein, 49 aa, Homo sapiens
  SOURCE 1..49: /mol_type=“protein” /organism=“Homo sapiens”
ILAFQLCIVLGSLGCYCQDPYVKEAENLKKYFNAGHSDVADNGTLFLGI

SEQ ID NO: 10 - protein, 143 aa, Homo sapiens
  SOURCE 1..143: /mol_type=“protein” /organism=“Homo sapiens”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 11 - protein, 143 aa, Homo sapiens
  SOURCE 1..143: /mol_type=“protein” /organism=“Homo sapiens”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 12 - protein, 55 aa, Homo sapiens
  SOURCE 1..55: /mol_type=“protein” /organism=“Homo sapiens”
QSQIVSFYFKLFKNFKDDQSIQKSVETIKEDMNVKFFNSNKKKRDDFEKLTNYSV

SEQ ID NO: 13 - protein, 46 aa, Homo sapiens
  SOURCE 1..46: /mol_type=“protein” /organism=“Homo sapiens”
MNVKFFNSNKKKRDDFEKLTNYSVTDLNVQRKAIHELIQVMAELSP

SEQ ID NO: 14 - protein, 39 aa, Homo sapiens
  SOURCE 1..39: /mol_type=“protein” /organism=“Homo sapiens”
SVETIKEDMNVKFFNSNKKKRDDFEKLTNYSVTDLNVQR

SEQ ID NO: 15 - protein, 25 aa, Homo sapiens
  SOURCE 1..25: /mol_type=“protein” /organism=“Homo sapiens”
VTDLNVQRKAIHELIQVMAELSPAA

SEQ ID NO: 16 - protein, 41 aa, Homo sapiens
  SOURCE 1..41: /mol_type=“protein” /organism=“Homo sapiens”
SYILAFQLCIVLGSLGCYCQDPYVKEAENLKKYFNAGHSDV

SEQ ID NO: 17 - protein, 21 aa, Homo sapiens
  SOURCE 1..21: /mol_type=“protein” /organism=“Homo sapiens”
ETIKEDMNVKFFNSNKKKRDD

SEQ ID NO: 18 - protein, 144 aa, artificial sequences
  SOURCE 1..144: /mol_type=“protein” /note=“recombinant human IFN gamma” /organism=“artificial sequences”
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 19 - protein, 144 aa, artificial sequences [sequence omitted]

SEQ ID NO: 20 - protein, 189 aa, Homo sapiens
  SOURCE 1..189: /mol_type=“protein” /organism=“Homo sapiens”
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 21 - protein, 188 aa, Homo sapiens [sequence omitted]

SEQ ID NO: 22 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 23 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 24 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 25 - protein, 189 aa, Homo sapiens
  SOURCE 1..189: /mol_type=“protein” /organism=“Homo sapiens”
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 26 - protein, 189 aa, Homo sapiens
  SOURCE 1..189: /mol_type=“protein” /organism=“Homo sapiens”
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 27 - protein, 189 aa, Homo sapiens
  SOURCE 1..189: /mol_type=“protein” /organism=“Homo sapiens”
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 28 - protein, 189 aa, Homo sapiens
  SOURCE 1..189: /mol_type=“protein” /organism=“Homo sapiens”
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 29 - protein, 189 aa, Homo sapiens
  SOURCE 1..189: /mol_type=“protein” /organism=“Homo sapiens”
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 30 - protein, 233 aa, Homo sapiens
  SOURCE 1..233: /mol_type=“protein” /organism=“Homo sapiens”
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 31 - protein, 5 aa, Homo sapiens
  SOURCE 1..5: /mol_type=“protein” /organism=“Homo sapiens”
PSDKP

SEQ ID NO: 32 - protein, 5 aa, Homo sapiens
  SOURCE 1..5: /mol_type=“protein” /organism=“Homo sapiens”
VANPQ

SEQ ID NO: 33 - protein, 135 aa, Homo sapiens
  SOURCE 1..135: /mol_type=“protein” /organism=“Homo sapiens”
RDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGV

SEQ ID NO: 34 - protein, 17 aa, Homo sapiens
  SOURCE 1..17: /mol_type=“protein” /organism=“Homo sapiens”
VRSSSRTPSDKPVAHVV

SEQ ID NO: 35 - protein, 23 aa, Homo sapiens
  SOURCE 1..23: /mol_type=“protein” /organism=“Homo sapiens”
FLSLFSFLIVAGATTLFCLLHFG

SEQ ID NO: 36 - protein, 18 aa, Homo sapiens
  SOURCE 1..18: /mol_type=“protein” /organism=“Homo sapiens”
IGPQREEFPRDLSLISPL

SEQ ID NO: 37 - protein, 38 aa, Homo sapiens
  SOURCE 1..38: /mol_type=“protein” /organism=“Homo sapiens”
QLVVPSEGLYLIYSQVLFKGQGCPSTHVLLTHTISRIA

SEQ ID NO: 38 - protein, 15 aa, Homo sapiens
  SOURCE 1..15: /mol_type=“protein” /organism=“Homo sapiens”
PCQRETPEGAEAKPW

SEQ ID NO: 39 - protein, 41 aa, Homo sapiens
  SOURCE 1..41: /mol_type=“protein” /organism=“Homo sapiens”
SMIRDVELAEEALPKKTGGPQGSRRCLFLSLFSFLIVAGAT

SEQ ID NO: 40 - protein, 35 aa, Homo sapiens
  SOURCE 1..35: /mol_type=“protein” /organism=“Homo sapiens”
VLLTHTISRIAVSYQTKVNLLSAIKSPCQRETPEG

SEQ ID NO: 41 - protein, 157 aa, Homo sapiens [sequence omitted]

SEQ ID NO: 42 - protein, 235 aa, Homo sapiens
  SOURCE 1..235: /mol_type=“protein” /organism=“Homo sapiens”
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 43 - protein, 20 aa, Homo sapiens
  SOURCE 1..20: /mol_type=“protein” /organism=“Homo sapiens”
PLALLLHAARPSQFRVSPLD

SEQ ID NO: 44 - protein, 20 aa, Homo sapiens
  SOURCE 1..20: /mol_type=“protein” /organism=“Homo sapiens”
AEGLDTQRFSGKRLGDTFVL

SEQ ID NO: 45 - protein, 20 aa, Homo sapiens
  SOURCE 1..20: /mol_type=“protein” /organism=“Homo sapiens”
SIMYFSHFVPVFLPAKPTTT

SEQ ID NO: 46 - protein, 10 aa, Homo sapiens
  SOURCE 1..10: /mol_type=“protein” /organism=“Homo sapiens”
VITLYCNHRN

SEQ ID NO: 47 - protein, 15 aa, Homo sapiens
  SOURCE 1..15: /mol_type=“protein” /organism=“Homo sapiens”
VVKSGDKPSLSARYV

(19) United States
(12) Patent Application Publication
(10) Pub. No.: US 2013/0045237 A1
(43) Pub. Date: Feb. 21, 2013

(54) Combination pharmaceutical composition and methods of treating and preventing the infectious diseases

(71) Applicants: Oleg Iliich Epshtein, Moscow (RU); Sergey Alexandrovish Tarasov, Golitsyno (RU)

(72) Inventors: Oleg Iliich Epshtein, Moscow (RU); Sergey Alexandrovish Tarasov, Golitsyno (RU)

(21) Appl. No.: 13/135,899

(22) Filed: Jul. 15, 2011

(30) Foreign Application Priority Data
Aug. 6, 2010 (RU) 2010133041
Aug. 6, 2010 (RU) 2010133043
Aug. 6, 2010 (RU) 2010133047
Aug. 6, 2010 (RU) 2010133050
Aug. 6, 2010 (RU) 2010133051
Aug. 6, 2010 (RU) 2010133052
Aug. 6, 2010 (RU) 2010133053
Jul. 4, 2011 (RU) 2011127226

(51) Int. Cl. A61K 39/395; A61P 31/00; A61P 31/12; A61P 31/04; A61P 37/00; A61P 31/14; A61P 31/16; A61K 9/00; A61P 31/18

(52) U.S. Cl. 424/400; 424/158.1; 424/143.1

(57) ABSTRACT

The present invention relates to a combination pharmaceutical composition comprising a) an activated-potentiated form of an antibody to at least one cytokine and b) an activated-potentiated form of an antibody to at least one receptor, and methods of treating and preventing the infectious diseases, including bacterial infections caused by different infectious agents such as pseudotuberculosis, whooping cough, yersiniosis, pneumonitis of different etiology, and acute and chronic viral infections such as acute respiratory tract infections, influenza of different types, acute viral hepatitis A, B, C and other types of hepatitis, the diseases and conditions caused by HIV or associated with HIV, including AIDS.

### FIELD

The present invention relates to a pharmaceutical composition and method of treating and preventing the infectious diseases, including bacterial infections caused by different infectious agents such as pseudotuberculosis, whooping cough, yersiniosis, pneumonitis of different etiology, and acute and chronic viral infections such as acute respiratory tract infections, influenza of different types, acute viral hepatitis A, B, C and other types of hepatitis, the diseases and conditions caused by HIV or associated with HIV, including AIDS.

### BACKGROUND

The invention relates to the area of medicine and may be used for the treatment and preventing the infectious diseases, including bacterial infections caused by different infectious agents such as pseudotuberculosis, whooping cough, yersiniosis, pneumonitis of different etiology, and acute and chronic viral infections such as acute respiratory tract infections, influenza of different types, acute viral hepatitis A, B, C and other types of hepatitis, the diseases and conditions caused by HIV or associated with HIV, including AIDS.

Treatment of viral diseases based on ultra-low doses of antibodies to interferon is known in the art (RU 2192888 C1, A61K39/395, Nov. 20, 2002). However, the given medical product can be not effective enough for treatment of the diseases associated with HIV.

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of diseases of viral etiology. See U.S. Pat. No. 7,572,441, which is incorporated herein by reference in its entirety.

The present invention is directed to a pharmaceutical composition and methods of its use in treatment and preventing of the infectious diseases, including bacterial infections caused by different infectious agents such as pseudotuberculosis, whooping cough, yersiniosis, pneumonitis of different etiology, and acute and chronic viral infections such as acute respiratory tract infections, influenza of different types, acute viral hepatitis A, B, C and other types of hepatitis, the diseases and conditions caused by HIV or associated with HIV, including AIDS.

The solution to the existing problem is presented in form of a combination pharmaceutical composition for treatment and prophylaxis (prevetion) of infectious diseases, which comprises a) an activated-potentiated form of antibodies to cytokine and b) an activated-potentiated form of antibodies to receptor.

### BRIEF DESCRIPTION OF THE DRAWINGS

FIG. 1 shows body temperature after administration of ultra-low doses (ULD) antibodies to interferon gamma and ULD antibodies to CD4 and ULD antibodies to histamine as compared to administration of placebo.

FIG. 2 shows body temperature after administration of ultra-low doses (ULD) antibodies to interferon gamma and ULD antibodies to CD4 and ULD antibodies to histamine as compared to administration of Tamiflu®.

### SUMMARY

In one aspect, the invention provides a combination pharmaceutical composition comprising a) an activated-potentiated form of an antibody to at least one cytokine and b) an activated-potentiated form of an antibody to at least one receptor. In an embodiment, the pharmaceutical composition further comprises a solid carrier, wherein said activated-potentiated forms of antibodies are impregnated onto said solid carrier. In a variant, the pharmaceutical composition is in the form of a tablet.

Preferably, the pharmaceutical composition including said activated-potentiated forms of antibodies is in the form of a mixture of C12, C30, and C200 homeopathic dilutions. It is specifically contemplated that said mixture of C12, C30, and C200 homeopathic dilutions is impregnated onto a solid carrier.

The activated-potentiated forms of said antibodies may be activated-potentiated forms of a monoclonal, polyclonal or natural antibody. It is specifically contemplated that the activated-potentiated form of said antibodies is activated-potentiated form of a polyclonal antibody. The invention provides activated-potentiated forms of antibodies to antigen(s) having sequences described in the specification and claimed in the appended claims.

In a variant, the pharmaceutical composition includes activated-potentiated forms of antibodies prepared by successive centesimal dilutions coupled with shaking of every dilution. Vertical shaking is specifically contemplated.

In another aspect, the invention provides a method of treating and preventing the infectious diseases, said method comprising administering to a patient in need thereof a) an activated-potentiated form of an antibody to at least one cytokine and b) an activated-potentiated form of an antibody to at least one receptor. Preferably, the activated-potentiated forms of antibodies are administered in the form of pharmaceutical composition.

In an embodiment, the pharmaceutical composition is administered in the form of a solid oral dosage form which comprises a pharmaceutically acceptable carrier and an activated-potentiated form of an antibody to at least one cytokine and activated-potentiated form of an antibody to at least one receptor, said activated-potentiated forms impregnated onto said carrier. In a variant, said solid oral dosage form is a tablet. Variants and embodiments are provided.

In accordance with the method aspect of the invention, the pharmaceutical composition may be administered in one to three unit dosage forms, each of the dosage form being administered from once daily to six times daily. In a variant, the pharmaceutical composition is administered twice daily, each administration consisting of two oral dosage forms. In a variant, the pharmaceutical composition is administered in one to two unit dosage forms, each of the dosage forms being administered twice daily. In a variant, the pharmaceutical composition is administered in one to two unit dosage forms, each of the dosage forms being administered four times daily. All variants and embodiments described with respect to the composition aspect of the invention may be used with the method aspect of the invention.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³° and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³° and 100⁵° times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well-accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³° and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated-potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated’ form of the antibodies of the present invention is not attributable to the initial molecular form of the antibody. Preferred is the “activated-potentiated” form of antibody in liquid or solid form in which the concentration of the molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides a combination pharmaceutical composition that includes activated-potentiated form of antibodies to cytokine and activated-potentiated form of antibodies to receptor, prepared according to the homeopathic technology of potentiation by repeated, consistent dilution and intermediate external action of shaking as described in more detail herein below. The pharmaceutical composition of the invention is particularly useful in the treatment and prophylaxis of the infectious diseases, including bacterial infections caused by different infectious agents such as pseudotuberculosis, whooping cough, yersiniosis, pneumonitis of different etiology, and acute and chronic viral infections such as acute respiratory tract infections, flu of different types, acute viral hepatitis A, B, C and other types of hepatitis, the diseases and conditions caused by HIV or associated with HIV, including AIDS. As shown in the Examples, the pharmaceutical composition of the invention possesses unexpected synergetic therapeutic effect, which manifest itself in particular therapeutic effectiveness in treating and preventing the infectious diseases, including bacterial infections caused by different infectious agents such as pseudotuberculosis, whooping cough, yersiniosis, pneumonitis of different etiology, and acute and chronic viral infections such as acute respiratory tract infections, influenza of different types, acute viral hepatitis A, B, C and other types of hepatitis, the diseases and conditions caused by HIV or associated with HIV, including AIDS.

The pharmaceutical composition of the invention expands the arsenal of preparations available for the treatment prophylaxis of the infectious diseases, including bacterial infections and acute and chronic viral infections.

The combination pharmaceutical composition in accordance with this aspect of the invention may be in the liquid form or in solid form. Activated-potentiated form of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R.—2005—Vol. 14.—N 1-2. P. 33-55, both incorporated herein by reference.

Monoclonal antibodies may be obtained, e.g., by means of hybridoma technology. The initial stage of the process includes immunization based on the principles already developed in the course of polyclonal antisera preparation. Further stages of work involve the production of hybrid cells generating clones of antibodies with identical specificity. Their separate isolation is performed using the same methods as in the case of polyclonal antisera preparation.

Polyclonal antibodies may be obtained via active immunization of animals. For this purpose, for example, suitable animals (e.g. rabbits) receive a series of injections of the appropriate antigen (cytokine and receptor). The animals' immune system generates corresponding antibodies, which are collected from the animals in a known manner. This procedure enables preparation of a monospecific antibody-rich serum.

If desired, the serum containing antibodies may be purified, for example by using affine chromatography, fractionation by salt precipitation, or ion-exchange chromatography. The resulting purified, antibody-enriched serum may be used as a starting material for the preparation of the activated-potentiated form of the antibodies. The preferred concentration of the resulting initial solution of antibody in the solvent, preferably water or a water-ethyl alcohol mixture, ranges from about 0.5 to about 5.0 mg/ml.

The preferred procedure for preparing each component of the combination drug according to the present invention is the use of the mixture of three aqueous-alcohol dilutions of the primary matrix solution of antibodies diluted 100¹², 100³⁰ and 100⁵⁰ times, respectively, which is equivalent to centesimal homeopathic dilutions C12, C30, and C50 or diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the combination pharmaceutical composition of the invention is polyclonal, animal-raised antibody to the corresponding antigen. To obtain the activated-potentiated form of polyclonal antibodies to cytokine or receptor, the desired antigen may be injected as immunogen into a laboratory animal, preferably, rabbits.

Polyclonal antibodies to CD4 receptor may be obtained using the whole molecule of human CD4 receptor of the following sequence:

```
SEQ. ID. NO. 1
Met Asn Arg Gly Val Pro Phe Arg His Leu Leu Leu Val Leu Gln
  1 5 10 15
Leu Ala Leu Leu Pro Ala Ala Thr Gln Gly Lys Lys Val Val Leu
  16 20 25 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
```

The polyclonal antibodies to CD4 receptor can be obtained using a polypeptide fragment of CD4 receptor chosen, for example, from the following amino-acid sequences:

```
SEQ. ID. NO. 2
                                                                        Gly Lys Lys Val Val Leu
                                                                          26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 3
                                                Ile Gly Leu Gly Ile Phe Phe Cys Val
                                                412 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 4
                                                                        Gly Lys Lys Val Val Leu
                                                                          26 30
Gly Lys Lys Gly Asp Thr Val Glu Leu Thr Cys Thr Ala Ser Gln
31 35 40 45
Lys Lys Ser Ile Gln Phe His Trp Lys Asn Ser Asn Gln Ile Lys
46 50 55 60
SEQ. ID. NO. 5
                                                                                                                Asp
  91 95 100 105
Thr Tyr Ile Cys Glu Val Glu Asp Gln Lys Glu Glu Val Gln
106 110 115 119
SEQ. ID. NO. 6
                                                                        Lys Glu Glu Val Gln Leu
                                                                        115 120
Leu Val Phe Gly Leu Thr Ala Asn Ser Asp Thr His Leu Leu Gln
121 125 130 135
Gly Gln Ser Leu
136 139
```

The exemplary procedure for preparation of the starting polyclonal antibodies to CD4 receptor may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. or without NaN₃ at the temperature of −70° C. To separate the target antibodies to gamma interferon from the antiserum, the following solid phase absorption sequence is suitable:

10 ml of the antiserum of rabbits is diluted twofold with 0.15 M NaCl, after which 6.26 g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to a DEAE-cellulose column balanced by phosphate buffer. The antibody fraction is determined by measuring the optical density of the eluate at 280 nm.

The isolated crude antibodies are purified using affine chromatography method by attaching the obtained antibodies to CD4 antigen located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to CD4 receptor is 0.5 to 5.0 mg/ml, preferably, 2.0 to 3.0 mg/ml.

The polyclonal antibodies to gamma interferon may also be obtained by a similar methodology to the methodology described for CD4 receptor antibodies using an adjuvant. Polyclonal antibodies to gamma interferon may be obtained using the whole molecule of gamma interferon of the following sequence:

```
SEQ ID NO: 7
Met Lys Tyr Thr Ser Tyr Ile Leu Ala Phe Gln Leu Cys Ile Val
  1 5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

Polyclonal antibodies to gamma interferon may be obtained using the whole molecule of gamma interferon of the following sequence:

```
SEQ ID NO: 8
Met Lys Tyr Thr Ser Tyr Ile Leu Ala Phe Gln Leu Cys Ile Val
  1 5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
```

The use of gamma interferon fragments as antigen is also contemplated. The suitable sequence for such antigen is as follow:

```
SEQ ID NO: 9
                                                Ile Leu Ala Phe Gln Leu Cys Ile Val
                                                  7 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile
  46 50 55
SEQ ID NO: 10
                                                                Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
SEQ ID NO: 11
                                                                Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ ID NO: 12
                                                                Gln Ser Gln Ile Val Ser Phe
                                                                  69 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val
121 123
SEQ ID NO: 13
                                                                        Met Asn Val Lys Phe Phe
                                                                        100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro
136 140 145
SEQ ID NO: 14
        Ser Val Glu Thr Ile Lys Glu Asp Met Asn Val Lys Phe Phe
          92 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg
121 125 130
SEQ ID NO: 15
                Val Thr Asp Leu Asn Val Gln Arg Lys Ala Ile His Glu
                  123 125 130 135
Leu Ile Gln Val Met Ala Glu Leu Ser Pro Ala Ala
136 140 145 147
SEQ ID NO: 16
                                Ser Tyr Ile Leu Ala Phe Gln Leu Cys Ile Val
                                  5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
SEQ ID NO: 17
                        Glu Thr Ile Lys Glu Asp Met Asn Val Lys Phe Phe
                          94 100 105
Asn Ser Asn Lys Lys Lys Arg Asp Asp
106 110 114
```

Polyclonal antibodies to gamma interferon may be obtained using the molecule of recombinant gamma interferon of one of the following sequences:

```
SEQ ID NO: 18
                                                        Met Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ ID NO: 19
                                                        Met Gln Asp Pro Tyr Val Lys Glu
                                                                24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

The polyclonal antibodies to alpha interferon may also be obtained by a similar methodology to the methodology described for CD4 receptor antibodies using an adjuvant. Polyclonal antibodies to alpha interferon may be obtained using the whole molecule of human alpha interferon type 8 of the following sequence:

```
SEQ ID NO: 20
Met Ala Leu Thr Phe Tyr Leu Leu Val Ala Leu Val Val Leu Ser
  1 5 10 15
Tyr Lys Ser Phe Ser Ser Leu Gly Cys Asp Leu Pro Gln Thr His
  16 20 25 30
Ser Leu Gly Asn Arg Arg Ala Leu Ile Leu Leu Ala Gln Met Arg
  31 35 40 45
Arg Ile Ser Pro Phe Ser Cys Leu Lys Asp Arg His Asp Phe Glu
  46 50 55 60
Phe Pro Gln Glu Glu Phe Asp Asp Lys Gln Phe Gln Lys Ala Gln
  61 65 70 75
Ala Ile Ser Val Leu His Glu Met Ile Gln Gln Thr Phe Asn Leu
  76 80 85 90
Phe Ser Thr Lys Asp Ser Ser Ala Ala Leu Asp Glu Thr Leu Leu
  91 95 100 105
Asp Glu Phe Tyr Ile Glu Leu Asp Gln Gln Leu Asn Asp Leu Glu
106 110 115 120
Ser Cys Val Met Gln Glu Val Gly Val Ile Glu Ser Pro Leu Met
121 125 130 135
Tyr Glu Asp Ser Ile Leu Ala Val Arg Lys Tyr Phe Gln Arg Ile
136 140 145 150
Thr Leu Tyr Leu Thr Glu Lys Lys Tyr Ser Ser Cys Ala Trp Glu
151 155 160 165
Val Val Arg Ala Glu Ile Met Arg Ser Phe Ser Leu Ser Ile Asn
166 170 175 180
Leu Gln Lys Arg Leu Lys Ser Lys Glu
181 185 189
```

Polyclonal antibodies to alpha interferon may be obtained using the whole molecule of human alpha interferon type 2 of the following sequence: SEQ ID NO: 21

```
Met Ala Leu Thr Phe Ala Leu Leu Val Ala Leu Leu Val Leu Ser
  1 5 10 15
Cys Lys Ser Ser Cys Ser Val Gly Cys Asp Leu Pro Gln Thr His
  16 20 25 30
Ser Leu Gly Ser Arg Arg Thr Leu Met Leu Leu Ala Gln Met Arg
  31 35 40 45
Lys Ile Ser Leu Phe Ser Cys Leu Lys Asp Arg His Asp Phe Gly
  46 50 55 60
Phe Pro Gln Glu Glu Phe Gly Asn Gln Phe Gln Lys Ala Glu Thr
  61 65 70 75
Ile Pro Val Leu His Glu Met Ile Gln Gln Ile Phe Asn Leu Phe
  76 80 85 90
Ser Thr Lys Asp Ser Ser Ala Ala Trp Asp Glu Thr Leu Leu Asp
  91 95 100 105
Lys Phe Tyr Thr Glu Leu Tyr Gln Gln Leu Asn Asp Leu Glu Ala
106 110 115 120
Cys Val Ile Gln Gly Val Gly Val Thr Glu Thr Pro Leu Met Lys
121 125 130 135
Glu Asp Ser Ile Leu Ala Val Arg Lys Tyr Phe Gln Arg Ile Thr
136 140 145 150
Leu Tyr Leu Lys Glu Lys Lys Tyr Ser Pro Cys Ala Trp Glu Val
151 155 160 165
Val Arg Ala Glu Ile Met Arg Ser Phe Ser Leu Ser Thr Asn Leu
166 170 175 180
Gln Glu Ser Leu Arg Ser Lys Glu
181 185 188
```

Polyclonal antibodies to alpha interferon may be obtained using the whole molecule of human alpha interferon type 17 of the following sequence:

```
SEQ ID NO: 22
Met Ala Leu Ser Phe Ser Leu Leu Met Ala Val Leu Val Leu Ser
  1 5 10 15
Tyr Lys Ser Ile Cys Ser Leu Gly Cys Asp Leu Pro Gln Thr His
  16 20 25 30
Ser Leu Gly Asn Arg Arg Ala Leu Ile Leu Leu Ala Gln Met Gly
  31 35 40 45
Arg Ile Ser Pro Phe Ser Cys Leu Lys Asp Arg His Asp Phe Gly
  46 50 55 60
Leu Pro Gln Glu Glu Phe Asp Gly Asn Gln Phe Gln Lys Thr Gln
  61 65 70 75
Ala Ile Ser Val Leu His Glu Met Ile Gln Gln Thr Phe Asn Leu
  76 80 85 90
Phe Ser Thr Glu Asp Ser Ser Ala Ala Trp Glu Gln Ser Leu Leu
  91 95 100 105
Glu Lys Phe Ser Thr Glu Leu Tyr Gln Gln Leu Asn Asn Leu Glu
106 110 115 120
Ala Cys Val Ile Gln Glu Val Gly Met Glu Glu Thr Pro Leu Met
121 125 130 135
Asn Glu Asp Ser Ile Leu Ala Val Arg Lys Tyr Phe Gln Arg Ile
136 140 145 150
Thr Leu Tyr Leu Thr Glu Lys Lys Tyr Ser Pro Cys Ala Trp Glu
151 155 160 165
Val Val Arg Ala Glu Ile Met Arg Ser Leu Ser Phe Ser Thr Asn
166 170 175 180
Leu Gln Lys Ile Leu Arg Arg Lys Asp
181 185 189
```

Polyclonal antibodies to alpha interferon may be obtained using the whole molecule of human alpha interferon type 4 of the following sequence:

```
SEQ ID NO: 23
Met Ala Leu Ser Phe Ser Leu Leu Met Ala Val Leu Val Leu Ser
  1 5 10 15
Tyr Lys Ser Ile Cys Ser Leu Gly Cys Asp Leu Pro Gln Thr His
  16 20 25 30
Ser Leu Gly Asn Arg Arg Ala Leu Ile Leu Leu Ala Gln Met Gly
  31 35 40 45
Arg Ile Ser His Phe Ser Cys Leu Lys Asp Arg His Asp Phe Gly
  46 50 55 60
Phe Pro Glu Glu Glu Phe Asp Gly His Gln Phe Gln Lys Ala Gln
  61 65 70 75
Ala Ile Ser Val Leu His Glu Met Ile Gln Gln Thr Phe Asn Leu
  76 80 85 90
Phe Ser Thr Glu Asp Ser Ser Ala Ala Trp Glu Gln Ser Leu Leu
  91 95 100 105
Glu Lys Phe Ser Thr Glu Leu Tyr Gln Gln Leu Asn Asp Leu Glu
106 110 115 120
Ala Cys Val Ile Gln Glu Val Gly Val Glu Glu Thr Pro Leu Met
121 125 130 135
Asn Glu Asp Ser Ile Leu Ala Val Arg Lys Tyr Phe Gln Arg Ile
136 140 145 150
Thr Leu Tyr Leu Thr Glu Lys Lys Tyr Ser Pro Cys Ala Trp Glu
151 155 160 165
Val Val Arg Ala Glu Ile Met Arg Ser Leu Ser Phe Ser Thr Asn
166 170 175 180
Leu Gln Lys Arg Leu Arg Arg Lys Asp
181 185 189
```

Polyclonal antibodies to alpha interferon may be obtained using the whole molecule of human alpha interferon type 21 of the following sequence:

```
SEQ ID NO: 24
Met Ala Leu Ser Phe Ser Leu Leu Met Ala Val Leu Val Leu Ser
  1 5 10 15
Tyr Lys Ser Ile Cys Ser Leu Gly Cys Asp Leu Pro Gln Thr His
  16 20 25 30
Ser Leu Gly Asn Arg Arg Ala Leu Ile Leu Leu Ala Gln Met Gly
  31 35 40 45
Arg Ile Ser Pro Phe Ser Cys Leu Lys Asp Arg His Asp Phe Gly
  46 50 55 60
Phe Pro Gln Glu Glu Phe Asp Gly Asn Gln Phe Gln Lys Ala Gln
  61 65 70 75
Ala Ile Ser Val Leu His Glu Met Ile Gln Gln Thr Phe Asn Leu
  76 80 85 90
Phe Ser Thr Lys Asp Ser Ser Ala Thr Trp Glu Gln Ser Leu Leu
  91 95 100 105
Glu Lys Phe Ser Thr Glu Leu Asn Gln Gln Leu Asn Asp Leu Glu
106 110 115 120
Ala Cys Val Ile Gln Glu Val Gly Val Glu Glu Thr Pro Leu Met
121 125 130 135
Asn Val Asp Ser Ile Leu Ala Val Lys Lys Tyr Phe Gln Arg Ile
136 140 145 150
Thr Leu Tyr Leu Thr Glu Lys Lys Tyr Ser Pro Cys Ala Trp Glu
151 155 160 165
Val Val Arg Ala Glu Ile Met Arg Ser Phe Ser Leu Ser Lys Ile
166 170 175 180
Phe Gln Glu Arg Leu Arg Arg Lys Glu
181 185 189
```

Polyclonal antibodies to alpha interferon may be obtained using the whole molecule of human alpha interferon type1/13 of the following sequence:

```
SEQ ID NO: 25
Met Ala Ser Pro Phe Ala Leu Leu Met Val Leu Val Val Leu Ser
  1 5 10 15
Cys Lys Ser Ser Cys Ser Leu Gly Cys Asp Leu Pro Glu Thr His
  16 20 25 30
Ser Leu AspA sn Arg Arg Thr Leu Met Leu Leu Ala Gln Met Ser
  31 35 40 45
Arg Ile Ser Pro Ser Ser Cys Leu Met Asp Arg His Asp Phe Gly
  46 50 55 60
Phe Pro Gln Glu Glu Phe Asp Gly Asn Gln Phe Gln Lys Ala Pro
  61 65 70 75
Ala Ile Ser Val Leu His Glu Leu Ile Gln Gln Ile Phe Asn Leu
  76 80 85 90
Phe Thr Thr Lys Asp Ser Ser Ala Ala Trp Asp Glu Asp Leu Leu
  91 95 100 105
Asp Lys Phe Cys Thr Glu Leu Tyr Gln Gln Leu Asn Asp Leu Glu
106 110 115 120
Ala Cys Val Met Gln Glu Glu Arg Val Gly Glu Thr Pro Leu Met
121 125 130 135
Asn Ala Asp Ser Ile Leu Ala Val Lys Lys Tyr Phe Arg Arg Ile
136 140 145 150
Thr Leu Tyr Leu Thr Glu Lys Lys Tyr Ser Pro Cys Ala Trp Glu
151 155 160 165
Val Val Arg Ala Glu Ile Met Arg Ser Leu Ser LeuS er Thr Asn
166 170 175 180
Leu Gln Glu Arg Leu Arg Arg Lys Glu
181 185 189
```

Polyclonal antibodies to alpha interferon may be obtained using the whole molecule of human alpha interferon type 10 of the following sequence:

```
SEQ ID NO: 26
Met Ala Leu Ser Phe Ser Leu Leu Met Ala Val Leu Val Leu Ser
  1 5 10 15
Tyr Lys Ser Ile Cys Ser Leu Gly Cys Asp Leu Pro Gln Thr His
  16 20 25 30
Ser Leu Gly Asn Arg Arg Ala Leu Ile Leu Leu Gly Gln Met Gly
  31 35 40 45
Arg Ile Ser Pro Phe Ser Cys Leu Lys Asp Arg His Asp Phe Arg
  46 50 55 60
Ile Pro Gln Glu Glu Phe Asp Gly Asn Gln Phe Gln Lys Ala Gln
  61 65 70 75
Ala Ile Ser Val Leu His Glu Met Ile Gln Gln Thr Phe Asn Leu
  76 80 85 90
Phe Ser Thr Glu Asp Ser Ser Ala Ala Trp Glu Gln Ser Leu Leu
  91 95 100 105
Glu Lys Phe Ser Thr Glu Leu Tyr Gln Gln Leu Asn Asp Leu Glu
106 110 115 120
Ala Cys Val Ile Gln Glu Val Gly Val Glu Glu Thr Pro Leu Met
121 125 130 135
Asn Glu Asp Ser Ile Leu Ala Val Arg Lys Tyr Phe Gln Arg Ile
136 140 145 150
Thr Leu Tyr Leu Ile Glu Arg Lys Tyr Ser Pro Cys Ala Trp Glu
151 155 160 165
Val Val Arg Ala Glu Ile Met Arg Ser Leu Ser Phe Ser Thr Asn
166 170 175 180
Leu Gln Lys Arg Leu Arg Arg Lys Asp
181 185 189
```

Polyclonal antibodies to alpha interferon may be obtained using the whole molecule of human alpha interferon type 5 of the following sequence:

```
SEQ ID NO: 27
Met Ala Leu Pro Phe Val Leu Leu Met Ala Leu Val Val Leu Asn
  1 5 10 15
Cys Lys Ser Ile Cys Ser Leu Gly Cys Asp Leu Pro Gln Thr His
  16 20 25 30
Ser Leu Ser Asn Arg Arg Thr Leu Met Ile Met Ala Gln Met Gly
  31 35 40 45
Arg Ile Ser Pro Phe Ser Cys Leu Lys Asp Arg His Asp Phe Gly
  46 50 55 60
Phe Pro Gln Glu Glu Phe Asp Gly Asn Gln Phe Gln Lys Ala Gln
  61 65 70 75
Ala Ile Ser Val Leu His Glu Met Ile Gln Gln Thr Phe Asn Leu
  76 80 85 90
Phe Ser Thr Lys Asp Ser Ser Ala Thr Trp Asp Glu Thr Leu Leu
  91 95 100 105
Asp Lys Phe Tyr Thr Glu Leu Tyr Gln Gln Leu Asn Asp Leu Glu
106 110 115 120
Ala Cys Met Met Gln Glu Val Gly Val Glu Asp Thr Pro Leu Met
121 125 130 135
Asn Val Asp Ser Ile Leu Thr Val Arg Lys Tyr Phe Gln Arg Ile
136 140 145 150
Thr Leu Tyr Leu Thr Glu Lys Lys Tyr Ser Pro Cys Ala Trp Glu
151 155 160 165
Val Val Arg Ala Glu Ile Met Arg Ser Phe Ser Leu Ser Ala Asn
166 170 175 180
Leu Gln Glu Arg Leu Arg Arg Lys Glu
181 185 189
```

Polyclonal antibodies to alpha interferon may be obtained using the whole molecule of human alpha interferon type 7 of the following sequence:

```
SEQ ID NO: 28
Met Ala Arg Ser Phe Ser Leu Leu Met Val Val Leu Val Leu Ser
  1 5 10 15
Tyr Lys Ser Ile Cys Ser Leu Gly Cys Asp Leu Pro Gln Thr His
  16 20 25 30
Ser Leu Arg Asn Arg Arg Ala Leu Ile Leu Leu Ala Gln Met Gly
  31 35 40 45
Arg Ile Ser Pro Phe Ser Cys Leu Lys Asp Arg His Glu Phe Arg
  46 50 55 60
Phe Pro Glu Glu Glu Phe Asp Gly His Gln Phe Gln Lys Thr Gln
  61 65 70 75
Ala Ile Ser Val Leu His Glu Met Ile Gln Gln Thr Phe Asn Leu
  76 80 85 90
Phe Ser Thr Glu Asp Ser Ser Ala Ala Trp Glu Gln Ser Leu Leu
  91 95 100 105
Glu Lys Phe Ser Thr Glu Leu Tyr Gln Gln Leu Asn Asp Leu Glu
106 110 115 120
Ala Cys Val Ile Gln Glu Val Gly Val Glu Glu Thr Pro Leu Met
121 125 130 135
Asn Glu Asp Phe Ile Leu Ala Val Arg Lys Tyr Phe Gln Arg Ile
136 140 145 150
Thr Leu Tyr Leu Met Glu Lys Lys Tyr Ser Pro Cys Ala Trp Glu
151 155 160 165
Val Val Arg Ala Glu Ile Met Arg Ser Phe Ser Phe Ser Thr Asn
166 170 175 180
Leu Lys Lys Gly Leu Arg Arg Lys Asp
181 185 189
```

Polyclonal antibodies to alpha interferon may be obtained using the whole molecule of human alpha interferon type 14 of the following sequence:

```
SEQ ID NO: 29
Met Ala Leu Pro Phe Ala Leu Met Met Ala Leu Val Val Leu Ser
  1 5 10 15
Cys Lys Ser Ser Cys Ser Leu Gly Cys Asn Leu Ser Gln Thr His
  16 20 25 30
Ser Leu Asn Asn Arg Arg Thr Leu Met Leu Met Ala Gln Met Arg
  31 35 40 45
Arg Ile Ser Pro Phe Ser Cys Leu Lys Asp Arg His Asp Phe Glu
  46 50 55 60
Phe Pro Gln Glu Glu Phe Asp Gly Asn Gln Phe Gln Lys Ala Gln
  61 65 70 75
Ala Ile Ser Val Leu His Glu Met Met Gln Gln Thr Phe Asn Leu
  76 80 85 90
Phe Ser Thr Lys Asn Ser Ser Ala Ala Trp Asp Glu Thr Leu Leu
  91 95 100 105
Glu Lys Phe Tyr Ile Glu Leu Phe Gln Gln Met Asn Asp Leu Glu
106 110 115 120
Ala Cys Val Ile Gln Glu Val Gly Val Glu Glu Thr Pro Leu Met
121 125 130 135
Asn Glu Asp Ser Ile Leu Ala Val Lys Lys Tyr Phe Gln Arg Ile
136 140 145 150
Thr Leu Tyr Leu Met Glu Lys Lys Tyr Ser Pro Cys Ala Trp Glu
151 155 160 165
Val Val Arg Ala Glu Ile Met Arg Ser Leu Ser Phe Ser Thr Asn
166 170 175 180
Leu Gln Lys Arg Leu Arg Arg Lys Asp
181 185 189
```

The polyclonal antibodies to CD8 receptor may also be obtained by a similar methodology to the methodology described for CD4 receptor antibodies using an adjuvant. Polyclonal antibodies to CD8 receptor may be obtained using the whole molecule of CD8 receptor of the following sequence:

```
SEQ ID NO: 30
Met Ala Leu Pro Val Thr Ala Leu Leu Leu Pro Leu Ala Leu Leu
  1 5 10 15
Leu His Ala Ala Arg Pro Ser Gln Phe Arg Val Ser Pro Leu Asp
  16 20 25 30
Arg Thr Trp Asn Leu Gly Glu Thr Val Glu Leu Lys Cys Gln Val
  31 35 40 45
Leu Leu Ser Asn Pro Thr Ser Gly Cys Ser Trp Leu Phe Gln Pro
  46 50 55 60
Arg Gly Ala Ala Ala Ser Pro Thr Phe Leu Leu Tyr Leu Ser Gln
  61 65 70 75
Asn Lys Pro Lys Ala Ala Glu Gly Leu Asp Thr Gln Arg Phe Ser
  76 80 85 90
Gly Lys Arg Leu Gly Asp Thr Phe Val Leu Thr Leu Ser Asp Phe
  91 95 100 105
Arg Arg Glu Asn Glu Gly Tyr Tyr Phe Cys Ser Ala Leu Ser Asn
106 110 115 120
Ser Ile Met Tyr Phe Ser His Phe Val Pro Val Phe Leu Pro Ala
121 125 130 135
Lys Pro Thr Thr Thr Pro Ala Pro Arg Pro Pro Thr Pro Ala Pro
136 140 145 150
Thr Ile Ala Ser Gln Pro Leu Ser Leu Arg Pro Glu Ala Cys Arg
151 155 160 165
Pro Ala Ala Gly Gly Ala Val His Thr Arg Gly Leu Asp Phe Ala
166 170 175 180
Cys Asp Ile Tyr Ile Trp Ala Pro Leu Ala Gly Thr Cys Gly Val
181 185 190 195
Leu Leu Leu Ser Leu Val Ile Thr Leu Tyr Cys Asn His Arg Asn
196 200 205 210
Arg Arg Arg Val Cys Lys Cys Pro Arg Pro Val Val Lys Ser Gly
211 215 220 225
Asp Lys Pro Ser Leu Ser Ala Arg Tyr Val
226 230 235
```

The use of CD8 receptor fragments as antigen is also contemplated. The suitable sequences for such antigen are as follow:

```
SEQ ID NO: 31
                                                                                Pro Leu Ala Leu Leu
                                                                                11 15
Leu His Ala Ala Arg Pro Ser Gln Phe Arg Val Ser Pro Leu Asp
  16 20 25 30
SEQ ID NO: 32
                                        Ala Glu Gly Leu Asp Thr Gln Arg Phe Ser
                                          81 85 90
Gly Lys Arg Leu Gly Asp Thr Phe Val Leu
  91 95 100
SEQ ID NO: 33
Ser Ile Met Tyr Phe Ser His Phe Val Pro Val Phe Leu Pro Ala
121 125 130 135
Lys Pro Thr Thr Thr
136 140
SEQ ID NO: 34
                                        Val Ile Thr Leu Tyr Cys Asn His Arg Asn
                                        201 205 210
SEQ ID NO: 35
                                                                                Val Val Lys Ser Gly
                                                                                221 225
Asp Lys Pro Ser Leu Ser Ala Arg Tyr Val
226 230 235
```

Polyclonal antibodies to tumor necrosis factor alpha (TNF-α) may be obtained by the above-mentioned method of obtaining antibodies to CD4 receptor using a whole molecule of tumor necrosis factor alpha of the following sequence:

```
SEQ ID NO: 36
Met Ser Thr Glu Ser Met Ile Arg Asp Val Glu Leu Ala Glu Glu
  1 5 10 15
Ala Leu Pro Lys Lys Thr Gly Gly Pro Gln Gly Ser Arg Arg Cys
  16 20 25 30
Leu Phe Leu Ser Leu Phe Ser Phe Leu Ile Val Ala Gly Ala Thr
  31 35 40 45
Thr Leu Phe Cys Leu Leu His Phe Gly Val Ile Gly Pro Gln Arg
  46 50 55 60
Glu Glu Phe Pro Arg Asp Leu Ser Leu Ile Ser Pro Leu Ala Gln
  61 65 70 75
Ala Val Arg Ser Ser Ser Arg Thr Pro Ser Asp Lys Pro Val Ala
  76 80 85 90
His Val Val Ala Asn Pro Gln Ala Glu Gly Gln Leu Gln Trp Leu
  91 95 100 105
Asn Arg Arg Ala Asn Ala Leu Leu Ala Asn Gly Val Glu Leu Arg
106 110 115 120
Asp Asn Gln Leu Val Val Pro Ser Glu Gly Leu Tyr Leu Ile Tyr
121 125 130 135
Ser Gln Val Leu Phe Lys Gly Gln Gly Cys Pro Ser Thr His Val
136 140 145 150
Leu Leu Thr His Thr Ile Ser Arg Ile Ala Val Ser Tyr Gln Thr
151 155 160 165
Lys Val Asn Leu Leu Ser Ala Ile Lys Ser Pro Cys Gln Arg Glu
166 170 175 180
Thr Pro Glu Gly Ala Glu Ala Lys Pro Trp Tyr Glu Pro Ile Tyr
181 185 190 195
Leu Gly Gly Val Phe Gln Leu Glu Lys Gly Asp Arg Leu Ser Ala
196 200 205 210
Glu Ile Asn Arg Pro Asp Tyr Leu Asp Phe Ala Glu Ser Gly Gln
211 215 220 225
Val Tyr Phe Gly Ile Ile Ala Leu
226 230 233
```

To obtain polyclonal antibodies to tumor necrosis factor alpha (TNF-α), it is also possible to use a polypeptide fragment of the tumor necrosis factor, selected, for example, from the following sequences:

```
SEQ ID NO: 37
Pro Ser Asp Lys Pro
  84 88
SEQ ID NO: 38
Val Ala Asn Pro Gln
  93 97
SEQ ID NO: 39
                                Arg Asp Leu Ser Leu Ile Ser Pro Leu Ala Gln
                                  65 70 75
Ala Val Arg Ser Ser Ser Arg Thr Pro Ser Asp Lys Pro Val Ala
  76 80 85 90
His Val Val Ala Asn Pro Gln Ala Glu Gly Gln Leu Gln Trp Leu
  91 95 100 105
Asn Arg Arg Ala Asn Ala Leu Leu Ala Asn Gly Val Glu Leu Arg
106 110 115 120
Asp Asn Gln Leu Val Val Pro Ser Glu Gly Leu Tyr Leu Ile Tyr
121 125 130 135
Ser Gln Val Leu Phe Lys Gly Gln Gly Cys Pro Ser Thr His Val
136 140 145 150
Leu Leu Thr His Thr Ile Ser Arg Ile Ala Val Ser Tyr Gln Thr
151 155 160 165
Lys Val Asn Leu Leu Ser Ala Ile Lys Ser Pro Cys Gln Arg Glu
166 170 175 180
Thr Pro Glu Gly Ala Glu Ala Lys Pro Trp Tyr Glu Pro Ile Tyr
181 185 190 195
Leu Gly Gly Val
196 199
SEQ ID NO: 40
        Val Arg Ser Ser Ser Arg Thr Pro Ser Asp Lys Pro Val Ala
          77 80 85 90
His Val Val
  91 93
SEQ ID NO: 41
        Phe Leu Ser Leu Phe Ser Phe Leu Ile Val Ala Gly Ala Thr
          32 35 40 45
Thr Leu Phe Cys Leu Leu His Phe Gly
  46 50 54
SEQ ID NO: 42
                                                                                Ile Gly Pro Gln Arg
                                                                                  56 60
Glu Glu Phe Pro Arg Asp Leu Ser Leu Ile Ser Pro Leu
61 65 70 73
SEQ ID NO: 43
                Gln Leu Val Val Pro Ser Glu Gly Leu Tyr Leu Ile Tyr
                123 125 130 135
Ser Gln Val Leu Phe Lys Gly Gln Gly Cys Pro Ser Thr His Val
136 140 145 150
Leu Leu Thr His Thr Ile Ser Arg Ile Ala
151 155 160
SEQ ID NO: 44
                                                                                Pro Cys Gln Arg Glu
                                                                                176 180
Thr Pro Glu Gly Ala Glu Ala Lys Pro Trp
181 185 190
SEQ ID NO: 45
                                Ser Met Ile Arg Asp Val Glu Leu Ala Glu Glu
                                  5 10 15
Ala Leu Pro Lys Lys Thr Gly Gly Pro Gln Gly Ser Arg Arg Cys
  16 20 25 30
Leu Phe Leu Ser Leu Phe Ser Phe Leu Ile Val Ala Gly Ala Thr
  31 35 40 45
SEQ ID NO: 46
                                                                                                                Val
                                                                                                                150
Leu Leu Thr His Thr Ile Ser Arg Ile Ala Val Ser Tyr Gln Thr
151 155 160 165
Lys Val Asn Leu Leu Ser Ala Ile Lys Ser Pro Cys Gln Arg Glu
166 170 175 180
Thr Pro Glu Gly
181 184
SEQ ID NO 47
        Val Arg Ser Ser Ser Arg Thr Pro Ser Asp Lys Pro Val Ala
          77 80 85 90
His Val Val Ala Asn Pro Gln Ala Glu Gly Gln Leu Gln Trp Leu
  91 95 100 105
Asn Arg Arg Ala Asn Ala Leu Leu Ala Asn Gly Val Glu Leu Arg
106 110 115 120
Asp Asn Gln Leu Val Val Pro Ser Glu Gly Leu Tyr Leu Ile Tyr
121 125 130 135
Ser Gln Val Leu Phe Lys Gly Gln Gly Cys Pro Ser Thr His Val
136 140 145 150
Leu Leu Thr His Thr Ile Ser Arg Ile Ala Val Ser Tyr Gln Thr
151 155 160 165
Lys Val Asn Leu Leu Ser Ala Ile Lys Ser Pro Cys Gln Arg Glu
166 170 175 180
Thr Pro Glu Gly Ala Glu Ala Lys Pro Trp Tyr Glu Pro Ile Tyr
181 185 190 195
Leu Gly Gly Val Phe Gln Leu Glu Lys Gly Asp Arg Leu Ser Ala
196 200 205 210
Glu Ile Asn Arg Pro Asp Tyr Leu Asp Phe Ala Glu Ser Gly Gln
211 215 220 225
Val Tyr Phe Gly Ile Ile Ala Leu
226 230 233
```

Polyclonal antibodies to histamine, which is a biogenic amine (4(2-aminoethyl)-imidazole or beta-imidazolylethylamine with the chemical formula C₅H₉N₃), may be obtained by the above-mentioned method of obtaining antibodies to CD4 using adjuvant and industrially produced histamine dihydrochloride as immunogen (antigen) for immunization of rabbits.

The activated-potentiated form of an antibody to cytokine or receptor may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to CD4 receptor with the concentration of 3.0 mg/ml is diluted in 99 parts of neutral aqueous or aqueous-alcohol solvent (preferably, 15%-ethyl alcohol) and then vertically shaked many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to gamma interferon with the concentration of 3.0 mg/ml in 99 parts of a neutral solvent in different containers, which is equivalent to the centesimal homeopathic dilution C12. Similar procedures with the relevant dilution factor are performed to obtain dilutions C30, C50 and C 200 The intermediate dilutions may be tested in a desired biological model to check activity. The preferred activated-potentiated form for the composition of the invention are a mixture of C12, C30, and C50 dilutions or C12, C30 and C200 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D20, C30, C100 or C12, C30, C50 or C12, C30, C200, etc.), the efficiency of which is determined experimentally by testing the dilution in a suitable biological model, for example, in models described in the examples herein.

In the course of potentiation and concentration decrease, the vertical shaking may be substituted for external exposure to ultrasound, electromagnetic field or any similar external impact procedure accepted in the homeopathic art.

Preferably, the pharmaceutical composition of the invention may be in the form of a liquid or in the solid unit dosage form. The preferred liquid carrier is water or water-ethyl alcohol mixture.

The solid unit dosage form of the pharmaceutical composition of the invention may be prepared by impregnating a solid, pharmaceutically acceptable carrier with the mixture of the activated potentiated form aqueous or aqueous-alcohol solutions of active component. Alternatively, the carrier may be impregnated consecutively with each requisite dilution. Both orders of impregnation are acceptable.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies to at least one cytokine and activated potentiated form of antibodies to at least one receptor. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono-olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

To prepare the solid oral form, 100-300 μm granules of lactose are impregnated with aqueous or aqueous-alcoholic solutions of the activated-potentiated forms of antibodies in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Huttlin Pilotlab” by Huttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch-XL 400 tablet press) to form 150 to 500 mg round pills, preferably, 300 mg. After tableting, 300 mg pills are obtained that are saturated with aqueous-alcohol solution (3.0-6.0 mg/pill) of the activated-potentiated form of antibodies in the form of a mixture of centesimal homeopathic dilutions C12, C30, and C50 or a mixture of centesimal homeopathic dilutions C12, C30 and C200.

While the invention is not limited to any specific theory, it is believed that the activated potentiated form of the antibodies described herein do not contain the molecular form of the antibody in an amount sufficient to have biological activity attributed to such molecular form. The biological activity of the combination drug (pharmaceutical composition) of the invention is amply demonstrated in the appended examples.

Preferably, for the purpose of treatment, the combination of the invention is administered from once daily to six times daily, preferably twice daily or four times daily, each administration including one or three combination unit dosage forms.

The invention is further illustrated with reference to the appended non-limiting examples.

### EXAMPLES

### Example 1

Study of the effect of a complex preparation containing ultralow doses of activated-potentiated forms of polyclonal affinity purified rabbit antibodies to CD4 receptor (anti-CD4) and gamma interferon (anti-IFN-γ), obtained by super-dilution of initial matrix solution (concentration: 2.5 mg/ml) (100¹², 100³⁰, 100⁵⁰ times), equivalent to a mixture of centesimal homeopathic dilutions C12, C30, C50 (ratio: 1:1) (<<anti-CD4+anti-IFN-γ>>), as well as its components: activated-potentiated form of polyclonal affinity purified rabbit antibodies to CD4 receptor, purified on antigen, obtained by super-dilution of initial matrix solution (100¹², 100³⁰, 100⁵⁰ times, equivalent to a mixture of centesimal homeopathic dilution C12, C30, C50 (“anti-CD4”), and activated-potentiated form of polyclonal rabbit antibodies to gamma interferon, obtained by super-dilution of initial matrix solution (100¹², 100³⁰, 100⁵⁰ times), equivalent to a mixture of centesimal homeopathic dilution C12, C30, C50 (“anti-IFN-γ”) on in vitro on binding of standard ligand [³H]pentazocine to human recombinant σ1 receptor was evaluated using radioligand method. Potentiated distilled water (mixture of homeopathic dilutions C12+C30+C50) was used as test preparations control.

The sigma-1 (σ1) receptor—an intracellular one which is localized in the cells of central nervous system, the cells of the most of peripheral tissues and immune competent cells. This receptor via control of homeostasis of intracellular calcium regulates intracellular signaling events leading to activation of the corresponding transcription factors and transcription of a whole gene family coding in particular the factors of resistance to infectious agents and cytokines. In this regard, the ability of drugs to influence to the efficiency of interaction of ligands with sigma-1 receptor indicates the presence of antiviral and immunomodulating components in the spectrum of its pharmacological activity that allows to consider these preparations as effective ones for the treatment and prophylaxis of various infectious diseases.

During the test (to measure total binding) 20 μl of the complex preparation anti-CD4+anti-IFN-γ or 10 μl of anti-CD4 or 10 μl of anti-IFN-γ were added to the incubation medium. Thus, the quantity of ULD of anti-CD4+anti-IFN-γ transferred into the test basin when testing the complex preparation was identical to that of anti-CD4 and ULD of anti-IFN-γ tested as monopreparations, which allows for a comparison of the efficiency of the preparation to its separate components. 20 μl and 10 μl of potentiated water were transferred into the incubation medium.

Further, 160 μl (about 200 μg of protein) of Jurkat cell line membranes homogenate (human leukemic T-lymphocyte line), and finally, 20 μl of tritium-labeled radioligand [³H]pentazocine (15 nm) were transferred.

In order to measure non-specific binding, 20 μl of non-labeled ligand-haloperidol (10 μM) were transferred in the incubation medium instead of the preparations or potentiated water.

Radioactivity was measured using a scintillometer (Topcount, Packard) and scintillation blend (Microscint 0, Packard) following the incubation within 120 minutes at 22° C. in 50 mM Tris-HCl buffer (pH=7.4) and filtration using fiberglass filters (GF/B, Packard). Specific binding (during the test or control) was calculated as a difference between total (during the test or control) and non-specific binding.

Results are represented as percentage of specific binding inhibition in control (distilled water was used as control) (Table 1).

TABLE 1

| | | | | | % of |
|---|---|---|---|---|---|
| | | | | | radioligand |
| | Quantity | % of radioligand specific | | | binding |
| | per test | binding in control | | | inhibition |
| Test group | basin | 1^{st} test | 2^{nd} test | Average | in control |
| anti-CD4 + | 20 μl | 50.8 | 49.1 | 49.9 | 50.1 |
| anti-IFN-γ | | | | | |
| anti-CD4 | 10 μl | 74.0 | 76.2 | 75.1 | 24.9 |
| anti-IFN-γ | 10 μl | 158.9 | 149.8 | 154.3 | −54.3 |
| Potentiated | 20 μl | 98.1 | 75.8 | 86.9 | 13.1 |
| water | | | | | |
| Potentiated | 10 μl | 140.1 | 106.2 | 123.2 | −23.2 |
| water | | | | | |

Effect of the preparations and potentiated water on binding of standard ligand [³H]pentazocine to human recombinant σ 1 receptor
Note:
% of specific binding in control = (specific binding during the test/specific binding in control)* 100%;
% of specific binding inhibition in control = 100% − (specific binding during the test/specific binding in control) * 100%).

The results reflecting inhibition above 50% represents significant effects of the tested compounds; inhibition from 25% to 50% confirms mild to moderate effects; inhibition less than 25% is considered to be insignificant effect of the tested compound and is within background level.

Therefore, this test model showed that the complex preparation of anti-CD4+anti-IFN-γ is more efficient than its separate components (anti-CD4 and anti-IFN-γ) in inhibiting the binding of standard radioligand [³H]pentazocine to human recombinant σ1 receptor; anti-CD4, transferred into the test basin, namely 10 μl, inhibit the binding of standard radioligand [3H]pentazocine to human recombinant σ1 receptor, but the effect intensity is inferior to that of the complex preparation of anti-CD4+anti-IFN-γ; anti-IFN-γ, transferred into the test well, namely 10 μl, had no effect on the binding of standard radioligand [3H]pentazocine to human recombinant σ1 receptor; potentiated water, transferred into the test basin, namely 10 μl or 20 μl, had no effect on the binding of standard radioligand [3H]pentazocine to human recombinant σ1 receptor.

### Example 2

### Mononuclear Cells; Reverse Transcriptase; Mode “Treatment”

List of Acronyms:

TCID50 stands for 50% Tissue Culture Infective Dose

Evaluation of antiretroviral activity of complex medication that contains ultra-low doses of rabbit polyclonal antibodies to CD4 (mixture of homeopathic dilutions C12+C30+C50) and ultra-low doses of rabbit polyclonal antibodies to interferon gamma (mixture of homeopathic dilutions C12+C30+C50) in 1:1 ratio (hereinafter referred to as Complex drug) and components that form part of it (ultra-low doses of rabbit polyclonal antibodies to CD4 (mixture of homeopathic dilutions C12+C30+C50) (hereinafter referred to as ULD AB to CD4) and ultra-low doses of rabbit polyclonal antibodies to interferon gamma (mixture of homeopathic dilutions C12+C30+C50 (hereinafter referred to as ULD AB to IFN gamma)) was performed with use of mononuclear cells of peripheral blood of a human being infected with in vitro strain HIV-1-LAI. As comparative drug azidothymidine was used (Sigma—AZ169-100 mg, lot 107 K1578).

Mononuclear cells of peripheral blood of a human being were separated from blood of healthy seronegative donor through centrifugation in density gradient ficcol-gipaque. The cells were activated during 3 days with use of 1 mkg/ml phytohemagglutinin P and 5 ME/ml of recombinant interleukine-2 of a human being in medium RPMI1640 (DIFCO) with 10% fetal calf serum (complement was removed through heating during 45 minutes within temperature of 56° C.), 1% solution of antibiotics (PSN Gibco containing 50 μg/ml penicillin, 50 μg/ml streptomycin and 100 μ/ml neomycin).

For evaluation of antiretroviral activity combination medications were introduced to well 15-30 minutes after contamination of cells with strain HIV-1-LAI with dose of 100 TCID50 (50 mkt inoculums of strain HIV-1-LAI). On the 7^{th} day after infection of cells supernatant used for evaluation of influence of medications to inhibition of HIV replication was selected.

Before introduction to well containing 150 μl of cell culture medications were diluted with medium RPMI1640 (DIFCO) till final volume of 50 μl was reached. ULD AB to CD4 and ULD AB to IFN gamma were diluted in medium RPMI1640 (DIFCO) in 8 times (degree of dilution 1/4). So quantity of ULD AB to CD4 and ULD AB to IFN gamma being introduced to experimental well during testing of complex drug similar to the quantity of ULD AB to CD4 and ULD AB to IFN gamma tested as mono-component that allows to make comparison of efficiency of complex drug with its separate components. Azidothymidine was diluted with medium RPMI1640 (DIFCO) up to concentration of 8 nM was achieved.

Efficiency medications was defined on inhibition of HIV replication that was evaluated on enzymatic activity of HIV-reverse transcriptase in supernatants of macrophages of peripheral blood of a human being with use of HIV RT RetroSys production set INNOVAGEN (lot 10-059C). For calculation of % of inhibition of HIV replication as control supernatant of cells was used to which tested medications were not introduced (see Table 2).

TABLE 2
Antiretroviral activity of medications with use
of mononuclear cells of peripheral blood of a human
being infected in vitro with strain HIV-1-LAI

| | Degree of | Inhibition of |
|---|---|---|
| | dilution | enzymatic activity of |
| | in media | HIV-reverse transcriptase |
| | RPMI1640 | (% from control) |
| Medication | (DIFCO) | 7^{th} day |
| ULD AB to IFN gamma | ⅛ | 0 ± 5 |
| ULD AB to CD4 | ⅛ | 67 ± 22 |
| Complex drug (ULD AB | ¼ | 85 ± 1 |
| to IFN gamma and ULD | | |
| AB to CD4 in 1:1 ratio) | | |
| Azidothymidine (8 nM) | — | 58 ± 7 |

Thus, in conditions of this experimental model it is shown that antiretroviral activity of complex drug exceeds antiretroviral activity of its separate components (ULD AB to IFN gamma and ULD AB to CD4).

### Example 3

### Macrophages; Reverse Transcriptase; Mode “Prophylaxis”

List of Acronyms:

TCID50—dose infecting 50% cells of tissue culture.

Evaluation of antiretroviral activity of complex medication that contains ultra-low doses of rabbit polyclonal antibodies to CD4 (mixture of homeopathic dilutions C12+C30+C50) and ultra-low doses of rabbit polyclonal antibodies to interferon gamma (mixture of homeopathic dilutions C12+C30+C50) in 1:1 ratio (hereinafter referred to as Complex drug) and components that form part of it (ultra-low doses of rabbit polyclonal antibodies to CD4 (mixture of homeopathic dilutions C12+C30+C50) (hereinafter referred to as ULD AB to CD4) and ultra-low doses of rabbit polyclonal antibodies to interferon gamma (mixture of homeopathic dilutions C12+C30+C50 (hereinafter referred to as ULD AB to IFN gamma)) was performed with use of macrophages that were received from mononuclear cells of peripheral blood of a human being infected with in vitro strain HIV-1-LAI. As comparative drug azidothymidine was used (Sigma—AZ169-100 mg, lot 107 K1578).

Macrophages of donor peripheral blood received from mononuclear cells of human peripheral blood were isolated from blood of two healthy seronegative donors through centrifugation in density gradient ficcol-gipaque. Mononuclear cells of human peripheral blood were grown for 3 days in medium RPMI1640 (DIFCO) that was added with 10% fetal calf serum (complement was removed through heating during 45 minutes within temperature of 56° C.), 1% solution of antibiotics (PSN Gibco containing 50 μg/ml penicillin, 50 μg/ml streptomycin and 100 μg/ml neomycin), 15 ng/ml GM-CSF (granulocyte macrophagal colony-stimulating factor). Then the cells were placed to culture plates (150000 cells/well in 48-well plate), grown during 7 days together with 1 ng/ml GM-CSF (granulocyte macrophagal colony-stimulating factor) and 10 ng/ml M-CSF (macrophagal colony-stimulating factor) so that the cells could be completely differentiated to macrophages.

For evaluation of antiretroviral activity combination medications were introduced to well 24 hours before contamination of cells with strain HIV-1-LAI with dose of 1000 TCID50 (100 mkl inoculums of strain HIV-1-Ba-L) and on the 3^{rd}, 7^{th}, 10^{th}, 14^{th}, 17^{tH} day after contamination. On the 3^{rd},7^{th}, 10^{th}, 14^{th}, 17^{th} day after infection of cells supernatant used for evaluation of influence of medications to inhibition of HIV replication was selected.

Before introduction to well containing 750 μl of cell culture medications were diluted with medium RPMI1640 (DIFCO) up to final volume of 250 μl was reached. ULD AB to CD4 and ULD AB to IFN gamma were diluted in medium RPMI1640 (DIFCO) in 8 times (degree of dilution 1/4). So quantity of ULD AB to CD4 and ULD AB to IFN gamma being introduced to experimental well during testing of complex drug similar to the quantity of ULD AB to CD4 and ULD AB to IFN gamma tested as mono-component that allows to make comparison of efficiency of complex drug with its separate components. Azidothymidine was diluted with medium RPMI1640 (DIFCO) till concentration of 8 nM was achieved.

Efficiency medications was defined by inhibition of HIV replication that was evaluated on enzymatic activity of HIV-reverse transcriptase in supernatants of supernatant macrophages of peripheral blood of a human being with use of HIV RT RetroSys production set INNOVAGEN (lot 10-059C). For calculation of % of inhibition of HIV replication as control supernatant of cells was used to which tested medications or azidothymidine were not introduced (see table 3 and 4).

TABLE 3
Antiretroviral activity of medications with use
of macrophages of human peripheral blood (donor
No. 1) infected in vitro with strain HIV-1-Ba-L

| | Degree of | | | |
|---|---|---|---|---|
| | dilution | | | |
| | in media | Inhibition of enzymatic activity of HIV- | | |
| | RPMI1640 | reverse transcriptase (% from control) | | |
| Medication | (DIFCO) | 14^{th} day | 17^{th} day | 21^{st} day |
| ULD AB to IFN | ⅛ | 24 ± 4 | 24 ± 4 | 0 ± 0 |
| gamma | | | | |
| ULD AB to CD4 | ⅛ | 53 ± 13 | 37 ± 7 | 0 ± 0 |
| Complex drug | ¼ | 69 ± 1 | 74 ± 9 | 37 ± 3 |
| (ULD AB to IFN | | | | |
| gamma and ULD | | | | |
| AB to CD4 in | | | | |
| 1:1 ratio) | | | | |
| Azidothymidine | — | 97 ± 1 | 97 ± 0 | 98 ± 2 |
| (8 nM) | | | | |

TABLE 4
Antiretroviral activity of medications with use
of macrophages of human peripheral blood (donor
No. 2) infected in vitro with strain HIV-1-Ba-L

| | Degree of | | | |
|---|---|---|---|---|
| | dilution | | | |
| | in media | Inhibition of enzymatic activity of HIV- | | |
| | RPMI1640 | reverse transcriptase (% from control) | | |
| Medication | (DIFCO) | 14^{th} day | 17^{th} day | 21^{st} day |
| ULD AB to IFN | ⅛ | 39 ± 20 | 0 ± 0 | 0 ± 0 |
| gamma | | | | |
| ULD AB to CD4 | ⅛ | 0 ± 0 | 0 ± 0 | 0 ± 0 |
| Complex drug | ¼ | 50 ± 5 | 42 ± 4 | 30 ± 6 |
| (ULD AB to IFN | | | | |
| gamma and ULD | | | | |
| AB to CD4 in | | | | |
| 1:1 ratio) | | | | |
| Azidothymidine | — | 82 ± 2 | 54 ± 1 | 41 ± 1 |
| (8 nM) | | | | |

So in conditions of this experimental model it is shown that:
1. Antiretroviral activity of complex medication exceeds antiretroviral activity of its separate components (ULD AB to IFN gamma and ULD AB to CD4).
2. Antiretroviral activity of complex medication is lasted during the whole experiment period in contrast to antiretroviral activity of its separate components (ULD AB to IFN gamma and ULD AB to CD4).
3. Only complex medication showed antiretroviral activity in in vitro model of infected macrophages of human peripheral blood received from different seronegative donors, which is the evidence of more pronounced antiretroviral effect of complex medication in comparison with its components (ULD AB to IFN gamma and ULD AB to CD4), antiretroviral activity of which was registered in in vitro model of infected macrophages of human peripheral blood received only from one seronegative donor.

### Example 4

### Mononuclear Cells; Reverse Transcriptase; Therapy Regimen

List of Abbreviations:

TCID50 stands for 50% Tissue Culture Infective Dose.

The assessment of antiretroviral activity of a complex product consisting of ultra low-dose rabbit polyclonal antibodies to interferon alpha, ultra low-dose rabbit polyclonal antibodies to interferon gamma, ultra low-dose rabbit polyclonal antibodies to CD4 and ultra low-dose rabbit polyclonal antibodies to CD8 as 1:1:1:1 ratio (a mixture of homoeopathic dilutions C12+C30+C50) (hereinafter referred to as the Complex product), was carried out using human peripheral blood mononuclear cells infected with the strain HIV-1LAI in vitro. Azidothymidine (Sigma—AZ169-100 mg, Lot 107 K1578) was used as a comparator product.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2 in RPMI1640 (DIFCO) medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin).

In order to assess antiretroviral activity the products were placed in a well 15-30 minutes after cells infection with the strain HIV-1-LAI at the dose of 100 TCID50 (50 μL inoculum of the strain HIV-1-LAI). Supernatant fluids used to assess the effect of products on the inhibition of HIV replication were also collected on day 7 after infection of cells.

Before placing in a well, which contained 150 μL of cell culture, the complex product was diluted with RPM11640 (DIFCO) medium at a 4-fold dilution (at a 1/4 dilution) to a final volume of 50 μL. Azidothymidine was diluted with RPMI1640 (DIFCO) medium to yield a 8 nM concentration.

The products' efficiency was established by the inhibition of HIV replication which was assessed by HIV-reverse transcriptase activity in the supernatant fluid from human peripheral blood mononuclear cells using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 5).

TABLE 5
Antiretroviral activity of the complex product
using human peripheral blood mononuclear cells
infected with the strain HIV-1-LAI in vitro

| | | Inhibition |
|---|---|---|
| | Medium | of HIV-reverse |
| | Dilution Ratio | transcriptase activity |
| | RPMI1640 | (% of control) |
| Product | (DIFCO) | Day 7 |
| Complex product (Ultra low- | ¼ | 81 ± 11 |
| dose antibodies to IFN-alpha, | | |
| Ultra low-dose antibodies | | |
| to IFN-gamma, Ultra low- | | |
| dose antibodies to CD4 and | | |
| Ultra low-dose antibodies | | |
| to CD8 as 1:1:1:1 ratio) | | |
| Azidothymidine (8 nM) | — | 58 ± 7 |

Thus, this experimental model demonstrated the antiretroviral activity of the complex product comprising ultra low-dose rabbit polyclonal antibodies to interferon alpha, ultra low-dose rabbit polyclonal antibodies to interferon gamma, ultra low-dose rabbit polyclonal antibodies to CD4 and ultra low-dose rabbit polyclonal antibodies to CD8 as 1:1:1:1 ratio (a mixture of homoeopathic dilutions C12+C30+C50).

### Example 5

### Mononuclear Cells; Nucleocapsid Protein p24; Prevention and Therapy Regimen

The assessment of antiretroviral activity of ultra low-dose of rabbit polyclonal antibodies to interferon-alpha (a mixture of homoeopathic dilutions C12+C30+C50), ultra low-dose of rabbit polyclonal antibodies to interferon-gamma (a mixture of homoeopathic dilutions C12+C30+C50) (ULD IFN-γ)), ultra low-dose of rabbit polyclonal antibodies to CD4 receptor (a mixture of homoeopathic dilutions C12+C30+C50) and ultra low-dose of rabbit polyclonal antibodies to CD8 receptor (a mixture of homoeopathic dilutions C12+C30+C50) (ULD Ab IFN-α+IFN-γ+CD4+CD8) was carried out using human peripheral blood mononuclear cells infected with the strain HIV-1 LAI in vitro.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2.

In order to assess antiretroviral activity the products were placed in a well containing 100 μL of activated mononuclears 24 hours before or 15 min after cell infection with the strain HIV-1-LAI at the dose of 100 TCID50 (50 μL inoculum of the strain HIV-1-LAI). Before adding to a well, ULD Ab IFN-α+IFN-γ+CD4+CD8 (12.5 μL) or reference azidotimidine (1000 nM) were mixed with RPMI1640 medium (DIFCO) to achive a final probe volume of 50 μL

The supernatant fluids were collected on day 7 after infection of cells. The products' activity was measured by the inhibition of HIV replication which was assessed by the level of core nucleocapsid protein p24 in the supernatant fluid from human peripheral blood mononuclear cells using Retrotek Elisa kit.

It was shown that ULD Ab IFN-α+IFN-γ+CD4+CD8 inhibited HIV replication by 94±6% when added to a well 24 hours before the infection, a and by 46±13% when added to a well 15 min after the infection of cells with the strain HIV-1 LAI. Azidotimidine at a dose of 1000 nM inhibited HIV replication by 99±0 and 99±1% added to a well 24 hours before and 15 min after the infection of cells with the strain HIV-1LAI, respectively.

Thus, this experimental model demonstrated the antiretroviral activity of ultra low-doses of rabbit polyclonal antibodies to ULD Ab IFN-α+IFN-γ+CD4+CD8 (a mixture of homoeopathic dilutions C12+C30+C50.

### Example 6

Investigation of efficiency of combined use of ultra-low doses of antibodies to interferon alpha (mixture of homeopathic dilutions C12+C30+C50) (hereinafter referred to as ULD AB to IFNalpha) and ultra-low doses of antibodies to CD4 (mixture of homeopathic dilutions C12+C30+C50) (hereinafter referred to as ULD AB to CD4) and ULD AB to IFNalpha and ULD AB to CD4 separately in the context of influenza infection at mice-female of the line Balb/c was performed on the basis of FSBI “SR1 of influenza” Ministry of health of social development of Russia (Saint Petersburg) in two stages. At the first stage efficiency of ULD AB to IFNalpha and ULD AB to CD4 was investigated, at the second stage efficiency of combined use of ULD AB to IFNalpha and ULD AB to CD4 (in 1:1 ratio) (hereinafter referred to as combination medication) was investigated. Both during testing of combination medication and during testing of ULD AB to IFNalpha and ULD AB to CD4 oseltamivir was used as comparative drug.

Infectious process was simulated through intranasal introduction of influenza virus A/California/07/2009swl (H1N1) with a dose 10LD50.

ULD AB to IFNalpha, ULD AB to CD4 and combination medication was intragastrically introduced to mice (n=20 in each group) at 0.2 ml/mouse twice a day (daily dose 0.4 ml/mouse) during 5 days before infection and during 10 days after infection. Additionally ULD AB to IFNalpha, ULD AB to CD4 and combination medication were added to drinking bowls of animals of corresponding experimental groups (free access was allowed).

Reference drug oseltamivir was intragastrically introduced to mice (n=20) twice a day with a dose of 10 mg/kg (daily dose 20 mg/kg) starting 1 hour before infection. Oseltamivir was introduced during 5 days after infection. During 4 days before infection and starting 6 days after infection distilled water at a dose of 0.2 ml/mouse twice a day (daily dose 0.4 ml/mouse) was intragastrically introduced instead of oseltamivir to mice of this experimental group. Distilled water was intragastrically introduced to mice of control group (n=20) twice a day at a dose of 0.2 ml/mouse (daily dose 0.4 ml/mouse). During the whole experiment period drinking bowls of animals of these two experimental groups contained distilled water (free access was allowed).

Efficacy of medications was evaluated by survival rate of animals. Results of study of antiviral activity of ULD AB to IFNalpha and ULD AB to CD4 (stage 1) see in Table 6, results of study of antiviral activity of combination medication (stage 2) see in Table 7. Statistical significance of differences between experimental groups and control (distilled water) was calculated with use of non-parametric chi-square criterion.

TABLE 6
Antiviral activity of ULD AB to IFNalpha and ULD AB to CD4 in the
model of influenza infection at female Balb/c mice infected through
intranasal introduction of influenza virus A/California/07/2009swl (H1N1)
with a dose of 10LD50 (10^{th} day after infection).

| | | | Difference between % of |
|---|---|---|---|
| | | | survival in the group that |
| | | Survival, | received medication and % |
| | Experimental | % | of survival in the group that |
| No. | group | 10LD50 | received distilled water |
| 1. | ULD AB to IFNalpha | 25 | +5% |
| 2. | ULD AB to CD4 | 30 | +10% |
| 3. | Oseltamivir | 80* | +60% |
| 4. | Distilled water | 20 | — |

*p < 0.05 vs control

TABLE 7
Antiviral activity combination medication containing ULD AB
to IFNalpha and ULD AB to CD4 in the model of influenza infection
at female Balb/c mice infected through intranasal introduction of
influenza virus A/California/07/2009swl (H1N1) with a dose of 10LD50
(10^{th} day after infection).

| | | | Difference between % of |
|---|---|---|---|
| | | | survival in the group that |
| | | Survival, | received medication and % |
| | Experimental | % | of survival in the group that |
| No. | group | 10LD50 | received distilled water |
| 1 | Combination medication | 30* | +25% |
| | (ULD AB to IFNalpha + | | |
| | ULD AB to CD4 in 1:1 | | |
| | ratio) | | |
| 2 | Oseltamivir | 70* | +65% |
| 3 | Distilled water | 5 | — |

*p < 0.05 vs control.

It is shown that survival of mice infected with influenza A/California/07/2009swl (H1N1) with a dose of 10LD50 was higher at the stage 1 than at the stage 2: survival in the group that received distilled water was 20% and 5% respectively; survival in the group of comparative drug oseltamivir was 80% and 70% respectively. It is the evidence of more expressed lethal effect induced through intranasal introduction of influenza virus A/California/07/2009swl (H1N1) with a dose of 10LD50, at the stage 2 of the study.

However, combination medication increased in 25% survival of experimental animals infected with influenza virus A/California/07/2009swl (H1N1) with a dose of 10LD50 as compared with control. Whereas survival in the group that received ULD AB to IFNalpha was only 5% higher than the survival in control group, and survival rate in the group received ULD AB to CD4 was only 10% higher than the survival in control group.

So as the result of performed study it was shown that combined use of ULD AB to IFNalpha and ULD AB to CD4 (combination medication) provides more pronounced antiviral effect than separate components, in spite of the fact that the dose of ULD AB to IFNalpha and ULD AB to CD4 as part of combination medication is twice lower than the dose of ULD AB to IFNalpha and ULD AB to CD4 tested as separate medications.

### Example 7

Investigation of efficiency of combined use of ultra-low doses of antibodies to tumor necrosis factor alpha (mixture of homeopathic dilutions C12+C30+C50) (hereinafter referred to as ULD Ab to TNFalpha) and ultra-low doses of antibodies to CD4 (mixture of homeopathic dilutions C12+C30+C50) (hereinafter referred to as ULD Ab to CD4) and ULD AB to TNFalpha and ULD AB to CD4 separately in the context of influenza infection at mice-female of the line Balb/c was performed on the basis of FSBI “SR1 of influenza” Ministry of health of social development of Russia (Saint Petersburg) in two stages. At the first stage efficiency of ULD Ab to TNFalpha and ULD Ab to CD4 was investigated, at the second stage efficiency of combined use of ULD AB to TNFalpha and ULD Ab to CD4 (in 1:1 ratio) (hereinafter referred to as combination medication) was investigated. Both during testing of combination medication and during testing of ULD AB to TNFalpha and ULD AB to CD4 oseltamivir was used as comparative drug.

Infectious process was simulated through intranasal introduction of influenza virus A/California/07/2009swl (H1N1) with a dose 10LD50.

ULD Ab to TNFalpha, ULD Ab to CD4 and combination medication was intragastrically introduced to mice (n=20 in each group) at 0.2 ml/mouse twice a day (daily dose 0.4 ml/mouse) during 5 days before infection and during 10 days after infection. Additionally ULD AB to TNFalpha, ULD AB to CD4 and combination medication were added to drinking bowls of animals of corresponding experimental groups (free access was allowed).

Reference drug oseltamivir was intragastrically introduced to mice (n=20) twice a day with a dose of 10 mg/kg (daily dose 20 mg/kg) starting 1 hour before infection. Oseltamivir was introduced during 5 days after infection. During 4 days before infection and starting 6 days after infection distilled water at a dose of 0.2 ml/mouse twice a day (daily dose 0.4 ml/mouse) was intragastrically introduced instead of oseltamivir to mice of this experimental group. Distilled water was intragastrically introduced to mice of control group (n=20) twice a day at a dose of 0.2 ml/mouse (daily dose 0.4 ml/mouse). During the whole experiment period drinking bowls of animals of these two experimental groups contained distilled water (free access was allowed).

Efficacy of medications was evaluated by survival rate of animals. Results of study of antiviral activity of ULD Ab to TNFalpha and ULD Ab to CD4 (stage 1) see in table 8, results of study of antiviral activity of combination medication (stage 2) see in table 9. Statistical significance of differences between experimental groups and control (distilled water) was calculated with use of non-parametric chi-square criterion.

TABLE 8
Antiviral activity of ULD Ab to TNFalpha and ULD Ab to CD4 in the
model of influenza infection at female Balb/c mice infected through
intranasal introduction of influenza virus A/California/07/2009swl (H1N1)
with a dose of 10LD50 (10^{th} day after infection).

| | | | Difference between % of |
|---|---|---|---|
| | | | survival in the group that |
| | | Survival, | received medication and % |
| | Experimental | % | of survival in the group that |
| No. | group | 10LD50 | received distilled water |
| 1. | ULD Ab to TNFalpha | 25 | +5% |
| 2. | ULD Ab to CD4 | 30 | +10% |
| 3. | Oseltamivir | 80* | +60% |
| 4. | Distilled water | 20 | — |

*p < 0.05 vs control

TABLE 9
Antiviral activity combination medication containing ULD Ab
to TNFalpha and ULD Ab to CD4 in the model of influenza infection
at female Balb/c mice infected through intranasal introduction of
influenza virus A/California/07/2009swl (H1N1) with a dose of 10LD50
(10^{th} day after infection).

| | | | Difference between % of |
|---|---|---|---|
| | | | survival in the group that |
| | | Survival, | received medication and % |
| | Experimental | % | of survival in the group that |
| No. | group | 10LD50 | received distilled water |
| 1. | Combination medication | 30* | +25% |
| | (ULD Ab to TNFalpha + | | |
| | ULD Ab to CD4 in 1:1 | | |
| | ratio) | | |
| 2. | Oseltamivir | 70* | +65% |
| 3. | Distilled water | 5 | — |

*p < 0.05 vs control.

It is shown that survival of mice infected with influenza A/California/07/2009swl (H1N1) with a dose of 10LD50 was higher at the stage 1 than at the stage 2: survival in the group that received distilled water was 20% and 5% respectively; survival in the group of comparative drug oseltamivir was 80% and 70% respectively. It is the evidence of more expressed lethal effect induced through intranasal introduction of influenza virus A/California/07/2009swl (H1N1) with a dose of 10LD50, at the stage 2 of the study.

However, combination medication increased in 25% survival of experimental animals infected with influenza virus A/California/07/2009swl (H1N1) with a dose of 10LD50 as compared with control. Whereas survival in the group that received ULD Ab to TNFalpha was only 5% higher than the survival in control group, and survival rate in the group received ULD Ab to CD4 was only 10% higher than the survival in control group.

So as the result of performed study it was shown that combined use of ULD Ab to TNFalpha and ULD Ab to CD4 (combination medication) provides more pronounced antiviral effect than separate components, in spite of the fact that the dose of ULD Ab to TNFalpha and ULD AB to CD4 as part of combination medication is twice lower than the dose of ULD Ab to TNFalpha and ULD AB to CD4 tested as separate medications.

### Example 8

Pharmaceutical composition (tablets) containing activated potentiated form of ultra-low doses (ULD) antibodies to interferon gamma (Ab IFNgamma), antibodies to CD4 (Ab CD4), antibodies to histamine (Ab His), impregnated onto lactose in the form of aqueous alcoholic solution of mixture of homeopathic dilutions C12, C30, C200 of each (Ab IFNgamma+Ab CD4+Ab to His) was used in the study.

In the double blind placebo-controlled study being conducted at present both men and women aged 18-60 years with viral URI's accompanied with intoxication, catarrh signs are enrolled. Patients with body temperature 37.8° C. and higher (provided that the temperature is registered at the onset of the disease), with the duration of the disease not exceeding 48 hours by the time of the therapy onset, not having severe complications were included in the study. Express test to detect influenza virus antigens was conducted. Patients with positive test results were not included in the study. Prior to the beginning of all the procedures the patients sign Informed consent to participate in the study. The patients were given diaries, in which body temperature twice daily, concomitant therapy, etc were registered. The patients receive Ab IFNgamma+Ab CD4+Ab His or placebo at a dose of 8 tablets daily on Day 1 and at a dose of 3 tablets daily on Days 2-5. If required the patients were allowed to take antipyretics. The intake of antiviral, immunomodulating, antihistamines and antibiotics is not allowed. Prior to start of therapy and at the last visit blood and urine samples are collected for assessment of laboratory parameters aimed at monitoring the safety of the conducted therapy. The overall therapy duration is 5 days, the duration of follow-up observation period is 2 days. Thus the duration of each patient's participation in the study is 7 days.

Time to reducing body temperature down to 37.0° C. and lower was considered as the therapy efficacy criterion; besides the number of antipyretics intakes was compared.

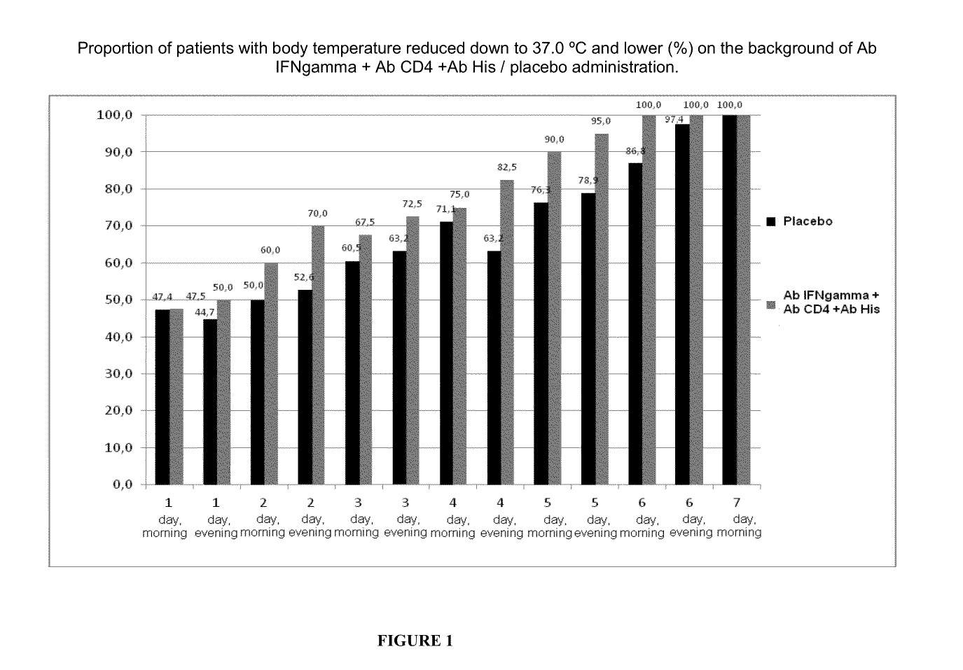

By the time when the analysis was conducted 78 patients finished the therapy (40 patients received Ab IFNgamma+Ab CD4+Ab His, 38 patients received placebo). The proportion of patients with the body temperature reduced down to 37.0° C. and lower are represented on FIG. 1. The Figure shows that Ab IFNgamma+Ab CD4+Ab His administration by the end of Day 2 from onset of the therapy resulted in 17.4% reduction in the patients' body temperature as compared to placebo group (p<0.05). At that the number of antipyretics intakes in the groups was significantly lower than in anti-Ab IFNgamma+Ab CD4+Ab His group (3.5±0.25 intake of antipyretics by the end of Day 2 of the treatment vs 3.9±0.32 in placebo group, p<0.05). Ab IFNgamma+Ab CD4+Ab His superiority over placebo group was seen as early as in the morning of Day 2 of the treatment and maintained all over therapy period.

Data of all 78 patients involved in the study and having finished the treatment in due terms were included in safety analysis; no discharge of patients were registered. Good drug tolerability was seen during the whole observation period. No adverse events related to Ab IFNgamma+Ab CD4+Ab His administration was registered. Blood tests conducted at the onset of the treatment and at the end of it did not show any pathologic deviations from norm. Urine analysis made on Day 1 and the last day of the study also did not reveal pathological changes in all patients.

When comparing the data with the results obtained during double blind placebo controlled randomized study of clinical efficacy and safety of Ab IFNgamma administration in influenza and other viral URI's conducted in 2005 (Influenza RI, RAMS, Saint-Petersburg, 2005) it was revealed that Ab IFNgamma+Ab CD4+Ab His reduces body temperature more effectively than Ab IFNgamma (FIG. 1, Table 10 and Table 11).

TABLE 10
Proportion of patients with body temperature reduced down to
37.0° C. and lower on the background Ab IFNgamma + Ab CD4 + Ab His/
placebo administration

| | | Day 1, | Day 1 | Day 2, | Day 2 | Day 3, | Day 3 | Day 4, |
|---|---|---|---|---|---|---|---|---|
| | | morning | evening | morning | evening | morning | evening | morning |
| Ab t Gl + Ab to Cd4 + | Total number of | 40 | 40 | 40 | 40 | 40 | 40 | 40 |
| Ab toH Ab IFNgamma + | patients | | | | | | | |
| Ab CD4 + Ab His, | The number of | 19 | 20 | 24 | 28 | 27 | 30 | 31 |
| n = 40 | patients with | | | | | | | |
| | normal | | | | | | | |
| | temperature | | | | | | | |
| | Proportion of | 47.5 | 50.0 | 60.0 | 70.0 | 67.5 | 75.0 | 77.5 |
| | patients with | | | | | | | |
| | normal | | | | | | | |
| | temperature, % | | | | | | | |
| Placebo n = 38 | Total number of | 38 | 38 | 38 | 38 | 38 | 38 | 38 |
| | patients | | | | | | | |
| | The number of | 18 | 17 | 19 | 20 | 23 | 24 | 27 |
| | patients with | | | | | | | |
| | normal | | | | | | | |
| | temperature | | | | | | | |
| | Proportion of | 47.4 | 44.7 | 50.0 | 52.6 | 60.5 | 63.2 | 71.1 |
| | patients with | | | | | | | |
| | normal | | | | | | | |
| | temperature, % | | | | | | | |
| Ab | Total number of | 30 | 30 | 30 | 30 | 30 | 30 | 30 |
| IFNgamma*, | patients | | | | | | | |
| n = 30 | The number of | 0 | 3 | 14 | 12 | 18 | 19 | 25 |
| | patients with | | | | | | | |
| | normal | | | | | | | |
| | temperature | | | | | | | |
| | Proportion of | 0 | 10.0 | 46.7 | 40.0 | 60.0 | 63.3 | 83.3 |
| | patients with | | | | | | | |
| | normal | | | | | | | |
| | temperature, % | | | | | | | |
| Placebo*, n = 30 | Total number of | 30 | 30 | 30 | 30 | 30 | 30 | 30 |
| | patients | | | | | | | |
| | The number of | 0 | 0 | 10 | 7 | 16 | 15 | 28 |
| | patients with | | | | | | | |
| | normal | | | | | | | |
| | temperature | | | | | | | |
| | Proportion of | 0 | 0 | 33.3 | 23.3 | 53.3 | 50.0 | 93.3 |
| | patients with | | | | | | | |
| | normal | | | | | | | |
| | temperature, % | | | | | | | |

| | | Day 4 | Day 5, | Day 5 | Day 6, | Day 6 | Day 7, |
|---|---|---|---|---|---|---|---|
| | | evening | morning | evening | morning | evening | morning |
| Ab t Gl + Ab to Cd4 + | Total number of | 40 | 40 | 40 | 40 | 40 | 40 |
| Ab toH Ab IFNgamma + | patients | | | | | | |
| Ab CD4 + Ab His, | The number of | 33 | 36 | 38 | 40 | 40 | 40 |
| n = 40 | patients with | | | | | | |
| | normal | | | | | | |
| | temperature | | | | | | |
| | Proportion of | 82.5 | 90.0 | 95.0 | 100.0 | 100.0 | 100.0 |
| | patients with | | | | | | |
| | normal | | | | | | |
| | temperature, % | | | | | | |
| Placebo n = 38 | Total number of | 38 | 38 | 38 | 38 | 38 | 38 |
| | patients | | | | | | |
| | The number of | 24 | 29 | 30 | 33 | 37 | 38 |
| | patients with | | | | | | |
| | normal | | | | | | |
| | temperature | | | | | | |
| | Proportion of | 63.2 | 76.3 | 78.9 | 86.8 | 97.4 | 100.0 |
| | patients with | | | | | | |
| | normal | | | | | | |
| | temperature, % | | | | | | |
| Ab | Total number of | 30 | 30 | 30 | 30 | 30 | 30 |
| IFNgamma*, | patients | | | | | | |
| n = 30 | The number of | 29 | 29 | 30 | 29 | 30 | 29 |
| | patients with | | | | | | |
| | normal | | | | | | |
| | temperature | | | | | | |
| | Proportion of | 96.7 | 96.7 | 100 | 96.7 | 100 | 96.7 |
| | patients with | | | | | | |
| | normal | | | | | | |
| | temperature, % | | | | | | |
| Placebo*, n = 30 | Total number of | 30 | 30 | 30 | 30 | 30 | 30 |
| | patients | | | | | | |
| | The number of | 28 | 28 | 30 | 30 | 30 | 30 |
| | patients with | | | | | | |
| | normal | | | | | | |
| | temperature | | | | | | |
| | Proportion of | 93.3 | 93.3 | 100 | 100 | 100 | 100 |
| | patients with | | | | | | |
| | normal | | | | | | |
| | temperature, % | | | | | | |

*According to the results of double blind placebo controlled randomized study of clinical efficacy and safety of Ab IFNgamma administration in influenza and other viral URI's conducted in 2005 (Influenza RI, RAMS, Saint-Petersburg, 2005)

TABLE 11
Mean values of body temperature in patients depending on
treatment groups, ° C., M ± SD

| | Day 1, | Day 1 | Day 2, | Day 2 | Day 3, | Day 3 | Day 4, |
|---|---|---|---|---|---|---|---|
| | morning | evening | morning | evening | morning | evening | morning |
| Ab | 37.5 ± 0.54 | 37.7 ± 0.56 | 37.2 ± 0.67 | 37.1 ± 0.53 | 36.8 ± 0.43 | 36.8 ± 0.49 | 36.7 ± 0.31 |
| IFNgamma + | | | | | | | |
| Ab CD4 + Ab | | | | | | | |
| His, n = 40 | | | | | | | |
| Placebo, | 37.6 ± 0.71 | 37.6 ± 0.63 | 37.2 ± 0.48 | 37.1 ± 0.49 | 36.9 ± 0.41 | 36.9 ± 0.36 | 36.8 ± 0.49 |
| n = 38 | | | | | | | |
| Ab | 38.1 ± 0.62 | 38.0 ± 0.58 | 37.4 ± 0.80 | 37.3 ± 0.61 | 37.1 ± 0.50 | 37.0 ± 0.47 | 36.8 ± 0.35 |
| IFNgamma, | | | | | | | |
| n = 30 | | | | | | | |
| Placebo,* | 38.0 ± 0.48 | 38.0 ± 0.50 | 37.4 ± 0.60 | 37.4 ± 0.47 | 37.0 ± 0.37 | 37.0 ± 0.42 | 36.8 ± 0.23 |
| n = 30 | | | | | | | |

| | Day 4 | Day 5, | Day 5 | Day 6, | Day 6 | Day 7, |
|---|---|---|---|---|---|---|
| | evening | morning | evening | morning | evening | morning |
| Ab | 36.6 ± 0.33 | 36.6 ± 0.25 | 36.6 ± 0.23 | 36.6 ± 0.22 | 36.6 ± 0.15 | 36.6 ± 0.18 |
| IFNgamma + | | | | | | |
| Ab CD4 + Ab | | | | | | |
| His, n = 40 | | | | | | |
| Placebo, | 36.7 ± 0.37 | 36.6 ± 0.32 | 36.6 ± 0.21 | 36.6 ± 0.28 | 36.5 ± 0.18 | 36.5 ± 0.18 |
| n = 38 | | | | | | |
| Ab | 36.6 ± 0.32 | 36.6 ± 0.21 | 36.5 ± 0.26 | 36.6 ± 0.21 | 36.6 ± 0.26 | 36.6 ± 0.26 |
| IFNgamma, | | | | | | |
| n = 30 | | | | | | |
| Placebo,* | 36.6 ± 0.34 | 36.6 ± 0.28 | 36.6 ± 5.42 | 36.6 ± 0.21 | 36.5 ± 0.24 | 36.6 ± 0.18 |
| n = 30 | | | | | | |

*According to the results of double blind placebo controlled randomized study of clinical efficacy and safety of Ab IFNgamma administration in influenza and other viral URI's (Influenza RI, RAMS, Saint-Petersburg, 2005)

### Example 9

Pharmaceutical composition (tablets) containing activated potentiated forms of ultra-low doses (ULD) antibodies to interferon—gamma (Ab IFNgamma), antibodies to CD4 (Ab to CD4), antibodies to histamine (Ab to H is), impregnated onto lactose in the form of aqueous alcoholic mixture of homeopathic dilutions C12, C30, C200 of each (Ab IFNgamma+Ab CD4+Ab His) was used in the study.

In the open-label comparative controlled clinical study of Ab IFNgamma+Ab CD4+Ab His and Tamiflu® (F. Hoffmann-La Roche Ltd—Switzerland, Oseltamivir) study being conducted at present both men and women aged 18-60 years with influenza accompanied by intoxication, catarrh signs are enrolled. Patients with body temperature 37.8° C. and higher (provided that the temperature is registered at the onset of the disease), with the duration of the disease not exceeding 48 hours by the time of the therapy onset, not having severe complications were included in the study. Express test to detect influenza virus antigens was conducted. Patients with positive test results were included in the study. Prior to the beginning of all the procedures the patients sign Informed consent to participate in the study. The patients were given diaries, in which body temperature twice daily, concomitant therapy, etc were registered. The patients receive Ab IFNgamma+Ab CD4+Ab His at a dose of 8 tablets daily on Day 1 and at a dose of 3 tablets daily on Days 2-5 or or Tamiflu at a dose of 75 mg 2 TID according to patient's information leaflet. If required the patients were allowed to take antipyretics. The intake of antiviral, immunomodulating, antihistamines and antibiotics is not allowed. Prior to start of therapy and at the last visit blood and urine samples are collected for assessment of laboratory parameters aimed at monitoring the safety of the conducted therapy. The overall therapy duration is 5 days, the duration of follow-up observation period is 2 days. Thus the duration of each patient's participation in the study is 7 days.

Time to reducing body temperature down to 37.0° C. and lower was considered as the therapy efficacy criterion; besides the number of antipyretics intakes was compared.

By the time when the analysis was conducted 17 patients have finished the therapy (6 patients in Ab IFNgamma+Ab CD4+Ab His group and 11 patient in Oseltamivir group).

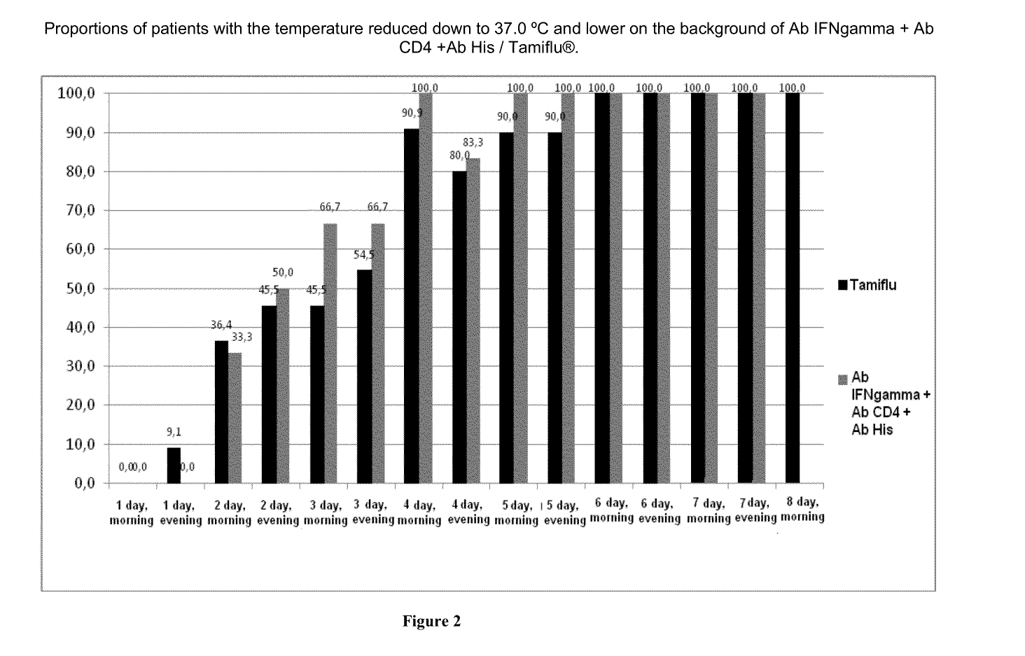

Proportions of patients with the body temperature reduced down to 37.0° C. and lower in the groups did not significantly differ in the course of therapy. As early as by Day 4 of the treatment patients of both groups practically recovered (see FIG. 2). As early as by Day 2 of the treatment in ⅓ of patients of both groups normalization of body temperature was registered. The difference in mean number of antipyretic intakes also was not significant and by the morning of Day 4 of the therapy was 7.6±0.8 in the group receiving Ab IFNgamma+Ab CD4+Ab His and 7.4±0.90 in Oseltamivir group respectively.

Data of all 17 patients involved in the study and having terminated the treatment in due terms were included in safety analysis; no discharge of patients were registered. Good drug tolerability was seen during the whole observation period. No adverse events related to Ab IFNgamma+Ab CD4+Ab His administration was registered. Blood tests conducted at the onset of the treatment and at the end of it did not show any pathologic deviations from norm. Urine analysis made on Day 1 and the last day of the study also did not reveal pathology in all patients.

When comparing the data with the results obtained during double blind placebo controlled randomized study of clinical efficacy and safety of Ab IFNgamma administration in influenza and other viral URI's conducted in 2005 (Influenza RI, RAMS, Saint-Petersburg, 2005) it was revealed that Ab IFNgamma+Ab CD4+Ab His reduces body temperature more effectively than Ab IFNgamma (FIG. 2, Table 12 and Table 13).

TABLE 12
Proportion of patients with body temperature reduced to 37.0° C.
and lower values on the background Ab IFNgamma + Ab CD4 + Ab His/
Oseltamivir administration

| | | Morning | Evening, | Morning | Evening, | Morning | Evening, | Morning | Evening, |
|---|---|---|---|---|---|---|---|---|---|
| | | Day 1 | Day 1 | Day 2 | Day 2 | Day 3 | Day 3 | Day 4 | Day 4 |
| Ab IFNgamma + Ab CD4 + | Total number of | 6 | 6 | 6 | 6 | 6 | 6 | 6 | 6 |
| Ab His, n = 6 | patients | | | | | | | | |
| | The number of | 0 | 0 | 2 | 3 | 4 | 4 | 6 | 5 |
| | patients with | | | | | | | | |
| | normal | | | | | | | | |
| | temperature | | | | | | | | |
| | Proportion of | 0 | 0 | 33.3 | 50.0 | 66.7 | 66.7 | 100.0 | 83.3 |
| | patients with | | | | | | | | |
| | normal temperature, % | | | | | | | | |
| [symbol] gamm [symbol] | Total number of | 11 | 11 | 11 | 11 | 11 | 11 | 11 | 10 |
| Oseltamivir, n = 11 | patients | | | | | | | | |
| | The number of | 0 | 1 | 4 | 5 | 5 | 6 | 10 | 8 |
| | patients with | | | | | | | | |
| | normal | | | | | | | | |
| | temperature | | | | | | | | |
| | Proportion of | 0 | 9.1 | 36.4 | 45.5 | 45.5 | 54.5 | 90.9 | 80.0 |
| | patients with | | | | | | | | |
| | normal temperature, % | | | | | | | | |
| | Total number of | 30 | 30 | 30 | 30 | 30 | 30 | 30 | 30 |
| | patients | | | | | | | | |
| | The number of | 0 | 3 | 14 | 12 | 18 | 19 | 25 | 29 |
| | patients with | | | | | | | | |
| | normal | | | | | | | | |
| | temperature | | | | | | | | |
| | Proportion of | 0 | 10.0 | 46.7 | 40.0 | 60.0 | 63.3 | 83.3 | 96.7 |
| | patients with | | | | | | | | |
| | normal temperature, % | | | | | | | | |
| Placebo*, n = 30 | Total number of | 30 | 30 | 30 | 30 | 30 | 30 | 30 | 30 |
| | patients | | | | | | | | |
| | The number of | 0 | 0 | 10 | 7 | 16 | 15 | 28 | 28 |
| | patients with | | | | | | | | |
| | normal | | | | | | | | |
| | temperature | | | | | | | | |
| | Proportion of | 0 | 0 | 33.3 | 23.3 | 53.3 | 50.0 | 93.3 | 93.3 |
| | patients with | | | | | | | | |
| | normal temperature, % | | | | | | | | |

| | | Morning | Evening, | Morning | Evening, | Morning | Evening, | Morning |
|---|---|---|---|---|---|---|---|---|
| | | Day 5 | Day 5 | Day 6 | Day 6 | Day 7 | Day 7 | Day 8 |
| Ab IFNgamma + Ab CD4 + | Total number of | 6 | 6 | 5 | 5 | 4 | 4 | N/A |
| Ab His, n = 6 | patients | | | | | | | |
| | The number of | 6 | 6 | 5 | 5 | 4 | 4 | N/A |
| | patients with | | | | | | | |
| | normal | | | | | | | |
| | temperature | | | | | | | |
| | Proportion of | 100.0 | 100.0 | 100.0 | 100.0 | 100.0 | 100.0 | N/A |
| | patients with | | | | | | | |
| | normal temperature, % | | | | | | | |
| [symbol] gamm [symbol] | Total number of | 10 | 10 | 9 | 7 | 5 | 4 | 3 |
| Oseltamivir, n = 11 | patients | | | | | | | |
| | The number of | 9 | 9 | 9 | 7 | 5 | 4 | 3 |
| | patients with | | | | | | | |
| | normal | | | | | | | |
| | temperature | | | | | | | |
| | Proportion of | 90.0 | 90.0 | 100.0 | 100.0 | 100.0 | 100.0 | 100.0 |
| | patients with | | | | | | | |
| | normal temperature, % | | | | | | | |
| | Total number of | 30 | 30 | 30 | 30 | 30 | 30 | 30 |
| | patients | | | | | | | |
| | The number of | 29 | 30 | 29 | 30 | 29 | 30 | 30 |
| | patients with | | | | | | | |
| | normal | | | | | | | |
| | temperature | | | | | | | |
| | Proportion of | 96.7 | 100 | 96.7 | 100 | 96.7 | 100 | 100 |
| | patients with | | | | | | | |
| | normal temperature, % | | | | | | | |
| Placebo*, n = 30 | Total number of | 30 | 30 | 30 | 30 | 30 | 30 | 30 |
| | patients | | | | | | | |
| | The number of | 28 | 30 | 30 | 30 | 30 | 30 | 30 |
| | patients with | | | | | | | |
| | normal | | | | | | | |
| | temperature | | | | | | | |
| | Proportion of | 93.3 | 100 | 100 | 100 | 100 | 100 | 100 |
| | patients with | | | | | | | |
| | normal temperature, % | | | | | | | |

*According to the results of double blind placebo controlled randomized study of clinical efficacy and safety of Ab IFNgamma administration in influenza and other viral URI's (Influenza RI, RAMS, Saint-Petersburg, 2005) [symbol]
indicates data missing or illegible when filed

TABLE 13
Mean values of body temperature in patients depending on
treatment groups, ° C., M ± SD

| | Morning | Evening, | Morning | Evening, | Morning | Evening, | Morning | Evening, |
|---|---|---|---|---|---|---|---|---|
| | Day 1 | Day 1 | Day 2 | Day 2 | Day 3 | Day 3 | Day 4 | Day 4 |
| Ab IFNgamma + | 38.5 ± 0.49 | 38.1 ± 0.62 | 37.2 ± 1.01 | 37.2 ± 0.67 | 36.5 ± 0.61 | 36.8 ± 0.47 | 36.5 ± 0.37 | 36.6 ± 0.46 |
| Ab IFNgamm [symbol] + | | | | | | | | |
| Ab CD4 + Ab | | | | | | | | |
| His, n = 6 | | | | | | | | |
| Oseltamivir, | 38.1 ± 0.82 | 37.3 ± 0.71 | 37.3 ± 0.72 | 36.9 ± 0.53 | 36.9 ± 0.47 | 36.7 ± 0.46 | 36.8 ± 0.37 | 36.5 ± 0.38 |
| n = 11 | | | | | | | | |
| Ab | 38.1 ± 0.62 | 38.0 ± 0.58 | 37.4 ± 0.80 | 37.3 ± 0.61 | 37.1 ± 0.50 | 37.0 ± 0.47 | 36.8 ± 0.35 | 36.6 ± 0.32 |
| IFNgamma*, | | | | | | | | |
| n = 30 | | | | | | | | |
| Placebo,* | 38.0 ± 0.48 | 38.0 ± 0.50 | 37.4 ± 0.60 | 37.4 ± 0.47 | 37.0 ± 0.37 | 37.0 ± 0.42 | 36.8 ± 0.23 | 36.6 ± 0.34 |
| n = 30 | | | | | | | | |

| | Morning | Evening, | Morning | Evening, | Morning | Evening, | Morning |
|---|---|---|---|---|---|---|---|
| | Day 5 | Day 5 | Day 6 | Day 6 | Day 7 | Day 7 | Day 8 |
| Ab IFNgamma + | 36.5 ± 0.26 | 36.6 ± 0.28 | 36.4 ± 0.35 | 36.5 ± 0.20 | 36.4 ± 0.26 | 36.5 ± 0.22 | ND |
| Ab IFNgamm [symbol] + | | | | | | | |
| Ab CD4 + Ab | | | | | | | |
| His, n = 6 | | | | | | | |
| Oseltamivir, | 36.7 ± 0.25 | 36.5 ± 0.21 | 36.6 ± 0.21 | 36.3 ± 0.19 | 36.5 ± 0.10 | 36.6 ± 0.12 | 36.5 ± 0.14 |
| n = 11 | | | | | | | |
| Ab | 36.6 ± 0.21 | 36.5 ± 0.26 | 36.6 ± 0.21 | 36.6 ± 0.26 | 36.6 ± 0.26 | 36.5 ± 0.27 | 36.5 ± 0.23 |
| IFNgamma*, | | | | | | | |
| n = 30 | | | | | | | |
| Placebo,* | 36.6 ± 0.28 | 36.6 ± 5.42 | 36.6 ± 0.21 | 36.5 ± 0.24 | 36.6 ± 0.18 | 36.5 ± 0.19 | 36.4 ± 0.21 |
| n = 30 | | | | | | | |

*According to the results of double blind placebo controlled randomized study of clinical efficacy and safety of Ab IFNgamma administration in influenza and other viral URI's (Influenza RI, RAMS, Saint-Petersburg, 2005) [symbol]
indicates data missing or illegible when filed

### Example 10

Pharmaceutical composition (tablets) containing activated potentiated forms of ultra-low doses (ULD) antibodies to interferon—gamma (Ab IFNgamma), antibodies to CD4 (Ab to CD4), antibodies to histamine (Ab to H is), impregnated onto lactose in the form of aqueous alcoholic mixture of homeopathic dilutions C12, C30, C200 of each (Ab IFNgamma+Ab CD4+Ab His) was used in the study.

In the present double blind placebo controlled study of efficacy and safety of Ab IFNgamma+Ab CD4+Ab His in viral URI's (Example 8) and in the present open-label comparative study of Ab IFNgamma+Ab CD4+Ab His efficacy and safety in influenza (Example 9) the number of complications including bacterial ones (bacterial pneumonia, tracheitis, otitis, glomerulonephritis, etc) developed on the background of acute infectious process were assessed in addition.

If the body defense system works properly infectious process can be arrested or localized, thus does not lead to the development of evident clinical symptoms, i.e. adequate defense reaction causes quick infectious agent inactivation, restoration of the body impaired functions and the recovery. Different situation can be seen in the subjects highly sensitive to infectious agent and lacking the proper mechanism of specific and non-specific defense (immunocompromized patients). In such cases increasingly replicated infectious agents and products of their interaction with epithelial and immune cells as well as damaged cells penetrate into blood causing the development of severe disease course, development of complications and potential poor outcome.

The use of Ab IFNgamma+Ab CD4+Ab His both in influenza and viral URI's caused considerable reduction in the frequency of bacterial complications as compared to placebo (Table 14) and therefore to reduction in antibacterial therapy. It seems that the drug inhibits the development of secondary immune deficit at the stage of recovery exerting immunomodulating effect and enhancing the body natural defense. The ability of Ab IFNgamma+Ab CD4+Ab His to reduce the frequency of bacterial complications development exceeded that of Ab IFNgamma.

TABLE 14
The frequency of bacterial complications

| | | Bacterial complications | | | |
|---|---|---|---|---|---|
| | | Oti- | Trache- | Pneu- | To- |
| | Number of | tis | itis | monia | tal |
| Drug | patients | n/% | n/% | n/% | n/% |
| Ab IFNgamma + | 40 | 0/0 | 1/2.5 | 0/0 | 1/2.5 |
| Ab CD4 + Ab His | | | | | |
| (viral URI's) | | | | | |
| Placebo | 38 | 3/7.9 | 7/18.4 | 0/0 | 10/26.3 |
| (viral URI's) | | | | | |
| Ab IFNgamma + | 6 | 0/0 | 0/0 | 0/0 | 0/0 |
| Ab CD4 + Ab His | | | | | |
| (influenza) | | | | | |
| Oseltamivir | 11 | 0/0 | 2/18.2 | 1/9.1 | 3/27.2 |
| (influenza) | | | | | |
| Ab IFNgamma | 30 | 1/3.3 | 2/6.7 | 0/0 | 3/10.0 |
| (influenza and | | | | | |
| viral URI's)* | | | | | |
| Placebo | 30 | 4/13.3 | 5/16.7 | 0/0 | 9/30 |
| (influenza and | | | | | |
| viral URI's)* | | | | | |

*According to the results of double blind placebo controlled randomized study of clinical efficacy and safety of Ab IFNgamma administration in influenza and other viral URI's (Influenza RI, RAMS, Saint- Petersburg, 2005)

### Example 11

To study the activity of pharmaceutical compositions for the treatment of patients of the group No. 1 tablets 300 mg impregnated onto pharmaceutical composition containing aqueous-alcoholic solutions (6 mg/tablet) of activated-potentiated forms of rabbit polyclonal affinity purified antibodies to human interferon gamma (anti-IFN-γ) and CD4 (anti-CD4) in ultra-low doses (ULD) obtained by means of ultra dilution of initial matrix solution in 100¹², 100³⁰ 100⁵⁰ times equal to mixture of centesimal homeopathic dilutions C12, C30, C50 were used; for treatment of patients of group No. 2 300 mg impregnated onto pharmaceutical composition containing aqueous-alcoholic solutions (6 mg/tablet) of activated-potentiated forms of rabbit polyclonal affinity purified antibodies to human interferon gamma (anti-IFN-γ) and CD4 (anti-CD4) and histamine (anti-His) in ultra-low doses (ULD) obtained by means of ultra dilution of initial matrix solution in 100¹², 100³⁰, 100⁵⁰ times equal to mixture of centesimal homeopathic dilutions C12, C30, C50 were used; for treatment of patients of group No. 3 tablets 300 mg impregnated onto pharmaceutical composition containing aqueous-alcoholic solutions (3 mg/tablet) of activated-potentiated forms of rabbit polyclonal affinity purified antibodies to human interferon gamma (anti-IFN-γ) in ultra-low doses (ULD) obtained by means of ultra dilution of initial matrix solution in 100¹², 100³⁰, 100⁵⁰ times equal to mixture of centesimal homeopathic dilutions C12, C30, C50 were used. Antiretroviral activity of pharmaceutical compositions ULD anti-IFN-γ+anti-CD4 and ULD anti-IFN-γ+anti-CD4+anti-His has been evaluated in the course of the open-label comparative clinical trial with participation of the human immunodeficiency virus (HIV) infected patients at Local Centre for Prevention and Fight Against AIDS and Infectious Diseases. The study included 97 patients (65 men and 32 women) aged 18-48 years old, with viral load of HIV-1 RNA ≧150 copies/ml in blood plasma and CD-4 lymphocyte counts ≧250 cells/pi (or ≧0.25×10⁹/l). Thirty four out of 97 study participants were treatment naïve patients. Sixty three out of 97 patients have been receiving antiretroviral therapy (ART) for one or two years. Patients with liver cirrhosis, viral hepatitis C, severe concomitant diseases in exacerbation period, pregnant women, as well as ones taking narcotic substances intravenously were not included in the study. The trial was carried out during autumn winter period when seasonal rise in influenza and acute respiratory viral infection is common.

Seventy five study participants were randomized into three groups prescribed either the study pharmaceutical compositions (groups No 1 and No 2) or reference pharmaceutical composition (group No 3) in a regimen corresponding to ARVI prophylaxis—1 tablet once a day for 6 weeks:
patients of group No 1 (n=25) were prescribed with ULD anti-IFN-γ+anti-CD4 (subgroup 1A: treatment naïve patients, n=12) or prescribed with ULD anti-IFN-γ+anti-CD4+ART (subgroup 1B, n=13);
patients of group No 2 (n=23) were prescribed with ULD anti-IFN-γ+anti-CD4+anti-His (subgroup 2A: treatment naïve patients, n=11) or prescribed with ULD anti-IFN-γ+anti-CD4+anti-His+ART (subgroup 2B, n=12);
patients of group No 3 (n=27) were prescribed with ULD anti-IFN-γ (subgroup 3A: treatment naïve patients, n=11) or prescribed with ULD anti-IFN-γ+ART (subgroup 3B, n=16).

The control group (group No 4, n=22) included patients who continued receiving ART alone in accordance with the earlier prescription (ART group).

At a baseline and after 6-week therapy viral load, CD4 [symbol] CD8 lymphocytes counts, CD4/CD8 immunoregulatory index were assayed in all the patients. To detect HIV-1 RNA copies in blood plasma the COBAS AMPLICOR HIV-1 MONITOR Kit (version 1.5 for automatic PCR-analyzer COBAS AMPLICOR, Roche, Switzerland) were used. Phenotyping of peripheral blood circulating lymphocytes was carried out on flow cytofluorometer FACSCount (Becton Dickinson, USA) using FACSCount Reagent Kit, which contain FITC PE fluorochrome-labeled antibodies to CD3, CD4, CD8.

Date on viral load (the number of copies of HCV RNA) presented in the table 15 as median (Me) and the range between first and third quartiles [Q1-Q3]. The study results indicate that 6-week treatment with ULD anti-IFN-γ+anti-CD4 decreased the number of RNA HIV-1 copies in 58% treatment naïve patients (in 7 out of 12 people of 1A subgroup), the average viral load decrease was 16.9%. Combination of ULD anti-IFN-γ+anti-CD4 and ART showed comparable efficacy, the number of HIV-1 RNA copies decreased in 62% of patients (in 8 out of 13 people in 1B subgroup), and the average viral load decrease from the baseline was 18.2%. Similar results were obtained in patients received ULD anti-IFN-γ+anti-CD4+anti-His: antiviral activity was registered in 55% HIV-infected treatment naïve patients (in 6 out of 11 people in 2A subgroup) and in 67% of patients receiving combination of ULD anti-IFN-γ+anti-CD4+anti-His and ART (in 8 out of 12 people in 2B subgroup); the average viral load decrease was 17.3% and 18.9% respectively. Antiretroviral activity observed in the first two groups was somewhat higher compared with the treatment outcome in control group. ULD anti-IFN-γmonotherapy for 6 weeks decreased the number of HIV-1 RNA copies in 36% treatment naïve patients (in 4 out of 11 people in 3A subgroup), the average viral load decrease was 9.5%. The combination of ULD anti-IFN-γ and ART improved the efficacy of therapy: the viral load decrease was registered in 50% of patients (in 8 out of 16 people in 3B subgroup), the average viral load decrease was 14.2%. In patients taking only ART (group No 4) the decrease in viral load were detected in 32% of patients (in 7 out of 22 patients) and an average viral load decrease 13.3%.

An assessment of circulating lymphocytes subpopulations during the study (Table 16) revealed more pronounced as compared to the control group increase in number of CD4 lymphocytes after 6-week therapy in ULD anti-IFN-γ+anti-CD4, ULD anti-IFN-γ+anti-CD4+anti-His and ULD anti-IFN-γ as a monotherapy in treatment naïve patients (groups 1A, 2A and 3A) or in combination with ART (subgroups 1B, 2B [symbol] 3B). The number of CD8 lymphocytes after 6-week therapy (without or in combination with ART) remained unchanged in all study groups. The positive dynamics in CD4-lymphocytes count in the course of the treatment resulted in increase in CD4/CD8 immunoregulatory index, which was most significant in the subgroups of patients taking ULD anti-IFN-γ+anti-CD4 and ULD anti-IFN-γ+anti-CD4+anti-His (without or in combination with ART, i.e. groups 1 and 2) and ULD anti-IFN-γ+ART (subgroup 3B).

No drugs-related adverse events were registered during the study, which evidences of their good tolerance. Absence of pathological variations in blood and urine analysis including markers of renal and hepatic insufficiency confirmed safety of the treatment.

Thus, the present study demonstrated antiretroviral activity of ULD anti-IFN-γ+anti-CD4 and ULD anti-IFN-γ+anti-CD4+anti-His pharmaceutical compositions, possibly mediated by the change in functional activity of CD4 receptors, which blocks HIV penetration into the cells, and also suppresses HIV replication inside the cell due to activation of transcription of mRNA of antiviral proteins. It was shown that the viral load decrease at the end 6-week course of ULD anti-IFN-γ+anti-CD4 and ULD anti-IFN-γ+anti-CD4+anti-His in the dose of 1 tablet a day was more pronounced compared to that of 6-week treatment with ULD anti-IFN-γ in the same dose or in patients continued receiving ART alone in accordance with the earlier prescription. The combination of ULD anti-IFN-γ+anti-CD4, ULD anti-IFN-γ+anti-CD4+anti-His or ULD anti-IFN-γmedication with ART somewhat increases the antiviral activity of the latter, which was revealed as the decrease of average viral load after 6 weeks in a larger proportion of patients.

The influence of ULD anti-IFN-γ+anti-CD4 and ULD anti-IFN-γ+anti-CD4+anti-His on CD4/CD8 lymphocytes ratio in HIV-infected patients (due to a decrease in the number of CD4 cells) was shown, which was most evident when combined with ART. Taking into consideration a simultaneous viral load decrease in patients taking ULD anti-IFN-γ+anti-CD4 and ULD anti-IFN-γ+anti-CD4+anti-His, one can assume that the increase in the number of CD4 cells is associated with population recruitment at the expense of healthy (non-infected) cells. Combination of ART with ULD anti-IFN-γ+anti-CD4, ULD anti-IFN-γ+anti-CD4+anti-His or ULD anti-IFN-γ more effectively recovers CD4/CD8 immunoregulatory index than ART alone does.

The observed antiretroviral activity of pharmaceutical compositions containing ULD anti-IFN-γ+anti-CD4 and ULD anti-IFN-γ+anti-CD4+anti-His makes it possible to use them for the treatment and prophylaxis of HIV infection both in treatment naïve HIV-infected patients and in patients taking ART.

TABLE 15
Viral Load Dynamics Depending on Therapy

| | |
|---|---|
| | Average Decrease of |
| Viral Load. copies/ml | Viral Load. % |

ULD anti-IFN-γ + anti-CD4 (Me [Q1-Q3])

| | | |
|---|---|---|
| Baseline | 5769 [368-62584] | 16.9 |
| After 6 weeks of treatment | 4575 [337-58526] | |

ART and ULD anti-IFN-γ + anti-CD4 (Me [Q1-Q3])

| | | |
|---|---|---|
| Baseline | 5238 [385-59695] | 18.2 |
| After 6 weeks of treatment | 4408 [320-50197] | |

ULD anti-IFN-γ + anti-CD4 + anti-H (Me [Q1-Q3])

| | | |
|---|---|---|
| Baseline | 5638 [385-61742] | 17.3 |
| After 6 weeks of treatment | 4754 [278-57426] | |

ART and ULD anti-IFN-γ + anti-CD4 + anti-H (Me [Q1-Q3])

| | | |
|---|---|---|
| Baseline | 5189 [350-59798] | 18.9 |
| After 6 weeks of treatment | 46108 [269-47987] | |

ULD anti-IFN-γ (Me [Q1-Q3])

| | | |
|---|---|---|
| Baseline | 5813 [150-33356] | 9.5 |
| After 6 weeks of treatment | 5786 [150-38359] | |

ART and ULD anti-IFN-γ (Me [Q1-Q3])

| | | |
|---|---|---|
| Baseline | 4680 [274-9838] | 14.2 |
| After 6 weeks of treatment | 4652 [272-8874] | |

ART (Me [Q1-Q3])

| | | |
|---|---|---|
| Baseline | 5547 [385-58996] | 13.3 |
| After 6 weeks of treatment | 5308 [338-57709] | |

TABLE 16
Circulating Lymphocytes Subpopulation
level in Patients of Study Groups

| | | |
|---|---|---|
| Observation Period | CD4. cl/mcl (M ± SE) | CD4/CD8 (M ± SE) |

ULD anti-IFN-γ + anti-CD4 (n = 12)

| | | |
|---|---|---|
| Baselint | 516 ± 33 | 0.46 ± 0.09 |
| After 6 weeks of treatment | 712 ± 24 | 0.58 ± 0.07* |

ART and ULD anti-IFN-γ + anti-CD4 (n = 13)

| | | |
|---|---|---|
| Baseline | 499 ± 41 | 0.50 ± 0.08 |
| After 6 weeks of treatment | 728 ± 29 | 0.60 ± 0.06* |

ULD anti-IFN-γ + anti-CD4 + anti-H (n = 11)

| | | |
|---|---|---|
| Baseline | 509 ± 45 | 0.49 ± 0.06 |
| After 6 weeks of treatment | 706 ± 27 | 0.58 ± 0.08* |

ART and ULD anti-IFN-γ + anti-CD4 + anti-H (n = 12)

| | | |
|---|---|---|
| Baseline | 521 ± 37 | 0.48 ± 0.09 |
| After 6 weeks of treatment | 734 ± 22 | 0.62 ± 0.10* |

ULD anti-IFN-γ (n = 11)

| | | |
|---|---|---|
| Baseline | 513 ± 98 | 0.38 ± 0.19 |
| After 6 weeks of treatment | 563 ± 26 | 0.44 ± 0.12 |

ART and ULD anti-IFN-γ (n = 16)

| | | |
|---|---|---|
| Initially | 491 ± 49 | 0.55 ± 0.06 |
| After 6 weeks of treatment | 623 ± 45 | 0.67 ± 0.05* |

ART (n = 22)

| | | |
|---|---|---|
| Initially | 510 ± 29 | 0.44 ± 0.06 |
| After 6 weeks of treatment | 595 ± 35 | 0.50 ± 0.12 |

*difference is significant vs baseline at p < 0.05

### Example 12

To study the activity of pharmaceutical compositions for the treatment of patients of the group No. 1 tablets 300 mg impregnated onto pharmaceutical composition containing aqueous-alcoholic solutions (6 mg/tablet) of activated-potentiated forms of rabbit polyclonal affinity purified antibodies to human interferon gamma (anti-IFN-γ) and CD4 (anti-CD4) in ultra-low doses (ULD) obtained by means of ultra dilution of initial matrix solution in 100¹², 100³⁰ 100⁵⁰ times equal to mixture of centesimal homeopathic dilutions C12, C30, C50 were used; for treatment of patients of group No. 2 300 mg impregnated onto pharmaceutical composition containing aqueous-alcoholic solutions (6 mg/tablet) of activated-potentiated forms of rabbit polyclonal affinity purified antibodies to human interferon gamma (anti-IFN-γ) and CD4 (anti-CD4) and histamine (anti-His) in ultra-low doses (ULD) obtained by means of ultra dilution of initial matrix solution in 100¹², 100³⁰, 100⁵⁰ times equal to mixture of centesimal homeopathic dilutions C12, C30, C50 were used; for treatment of patients of group No. 3 tablets 300 mg impregnated onto pharmaceutical composition containing aqueous-alcoholic solutions (3 mg/tablet) of activated-potentiated forms of rabbit polyclonal affinity purified antibodies to human interferon gamma (anti-IFN-γ) in ultra-low doses (ULD) obtained by means of ultra dilution of initial matrix solution in 100¹², 100³⁰, 100⁵⁰ times equal to mixture of centesimal homeopathic dilutions C12, C30, C50 were used.

Evaluation of efficacy of three pharmaceutical compositions containing ULD anti-IFN-γ+anti-CD4, ULD anti-IFN-γ+anti-CD4+anti-His and ULD anti-IFN-γ in the treatment of chronic viral hepatitis C was performed in the course of comparative parallel group study. Eighteen patients (14 men and 4 women) at the age of 27-52 were enrolled. Diagnosis of hepatitis C was confirmed by serum markers (anti-HVC and HCV RNA). All patients included to the study had 2^{nd} or 3^{rd} genotype HCV, mild slowly progressive course of chronic hepatitis C with low disease activity (serum aminotransferases <3-fold normal values or <100 U/I); none of the patients receive specific antiviral therapy before. The patients with positive result of serologic analysis for HIV, RW, anti-HCA, HBsAg or HBcorAg Ab, with cirrhosis, severe concomitant diseases at the stage of exacerbation, thalassemia or other hemoglobinopathy, alcoholic and\or medication/drug dependence, patients after transplantation of organs who constantly took immunosuppressive medications as well as pregnant women and lactating women were not included in the study. The patients of three study groups were given the pharmaceutical compositions according to the following regimen: 1 tablet three times a day for 24 weeks: patients of the 1^{st} group (n=5) -ULD anti-IFN-γ+anti-CD4; patients of the 2^{nd} group (n=4) -ULD anti-IFN-γ+anti-CD4+anti-His; patients of the 3^{rd} group (n=4)-ULD anti-IFN-γ. Control group consisted of 5 patients with persistent viremia and stable normal levels of aminotransferases (<20 U/I) received no specific therapy. During the study course regular examinations, control of viral load and laboratory rates were carried out, concomitant therapy was registered as well as undesirable adverse events. Therapy efficacy was assessed on week 24 by viral load with HCV RNA and activity of alanine-aminotransferase (ALT).

Date on viral load (the number of copies of HCV RNA) presented in the table as median (Me) and the range between first and third quartiles [Q1-Q3], evidenced positive effect of therapy in patients of groups 1-3 by the end of 24-week treatment. Intake of pharmaceutical composition of ULD anti-IFN-γ+anti-CD4 caused a reduction in the number of copies of HCV RNA in 2 out of 5 persons of the 1^{st} group and an average reduction of viral load was 75%. Similar results were obtained in patients administered with pharmaceutical composition of ULD anti-IFN-γ+anti-CD4+anti-His: its antiviral activity was registered in all patients (4 out of 4 subjects of the group 2), average reduction of viral load was 70%. Moreover, complete virus clearance was registered in 2 patients (one of group 1 and one of group 2) by the end of therapy. Antiviral activity of monocomponent ULD anti-IFN-γ was somewhat lower and a reduction in the number of copies of HCV RNA was recorded in 3 out of 4 patients of 3^{rd} group, an average reduction of viral load was 55%. In control group, no positive changes in viral load were revealed.

Antiviral activity of the studies pharmaceutical compositions was accompanied with positive changes in ALT level registered in patients of groups 1-3 by the end of 24-week therapy. Normalization of ALT level was found in 2 patients of ULD anti-IFN-γ+anti-CD4 group, in 1 patient of ULD anti-IFN-γ+anti-CD4+anti-His group and in 1 patient ULD anti-IFN-γ group. In 1 patient of control group ALT level exceeded upper border of norm (>20 U/I) due to an increase of viral load at the end of 24-week study period.

No drugs-related adverse events were registered during the study, which evidences of their good tolerance. Absence of pathological variations in blood and urine analysis including markers of renal and hepatic insufficiency confirmed safety of the treatment.

Thus, the study of efficacy and safety of pharmaceutical compositions containing ULD anti-IFN-γ+anti-CD4, ULD anti-IFN-γ+anti-CD4+anti-His and ULD anti-IFN-γ in patients with chronic hepatitis C were carried out. The strongest antiviral effect was registered for ULD anti-IFN-γ+anti-CD4, ULD anti-IFN-γ+anti-CD4+anti-His, which was confirmed by positive dynamics of viral load and viral clearance by the end of 24-week therapy in 2 patients. Antiviral efficacy of ULD anti-IFN-γ+anti-CD4, ULD anti-IFN-γ+anti-CD4+anti-His and ULD anti-IFN-γ was accompanied with a reduction of activity of chronic hepatitis C, which was confirmed by the reduction and even normaluzation of ALT level in some patients at the end of 24-week course of treatment.

TABLE 17
Dynamics of viral load in the study groups

| | |
|---|---|
| | Average reduction of |
| HCV RNA, copies/ml | viral load, % |

ULD anti-IFN-γ + anti-CD4 (Me [Q1-Q3])

| | | |
|---|---|---|
| Baseline | 66200 [450-181400] | 75 |
| 24-week treatment | 12500 [50-30560] | |

ULD anti-IFN-γ + anti-CD4 + anti-His (Me [Q1-Q3])

| | | |
|---|---|---|
| Baseline | 58900 [600-124500] | 70 |
| 24-week treatment | 15600 [50-45700] | |

ULD anti-IFN-γ (Me [Q1-Q3])

| | | |
|---|---|---|
| Baseline | 84700 [350-172800] | 55 |
| 24-week treatment | 22400 [150-58500] | |

Control group (Me [Q1-Q3])

| | | |
|---|---|---|
| Baseline | 79500 [300-155600] | — |
| 24-week treatment | 87900 [450-164300] | |

What is claimed is:

1. A combination pharmaceutical composition comprising a) an activated-potentiated form of an antibody to at least one cytokine and b) an activated-potentiated form of an antibody to at least one receptor.

2. The combination pharmaceutical composition of claim 1, wherein the activated-potentiated form of an antibody to at least one cytokine is prepared by successive centesimal dilutions coupled with shaking of every dilution.

3. The combination pharmaceutical composition of claim 1, wherein the activated-potentiated form of an antibody to at least one receptor is prepared by successive centesimal dilutions coupled with shaking of every dilution.

4. The combination pharmaceutical composition of claim 1, wherein the activated-potentiated form of an antibody to at least one cytokine is in the form of a mixture of C12, C30, and C50 homeopathic dilutions impregnated onto a solid carrier and the activated-potentiated form of an antibodies to at least one receptor is in the form of mixture of C12, C30, and C50 homeopathic dilutions impregnated onto said solid carrier.

5. The combination pharmaceutical composition of claim 1, wherein the activated-potentiated form of an antibody to at least one cytokine is in the form of a mixture of C12, C30, and C200 homeopathic dilutions impregnated onto a solid carrier and the activated-potentiated form of an antibodies to at least one receptor is in the form of mixture of C12, C30, and C200 homeopathic dilutions impregnated onto said solid carrier.

6. The combination pharmaceutical composition of claim 5, wherein said carrier is impregnated with a mixture of said dilutions.

7. The combination pharmaceutical composition of claim 1, wherein said antibody is a monoclonal, polyclonal or natural antibody.

8. The combination pharmaceutical composition of claim 7, wherein said antibody is a polyclonal antibody.

9. The combination pharmaceutical composition of claim 1, wherein said at least one cytokine is gamma interferon and wherein said at least one receptor is CD4 receptor.

10. The combination pharmaceutical composition of claim 9, further comprising an activated-potentiated form of an antibody to histamin.

11. The combination pharmaceutical composition of claim 1, wherein said at least one cytokine is gamma interferon and alpha interferon, and wherein said at least one receptor is CD4 receptor and CD8 receptor.

12. The combination pharmaceutical composition of claim 1, wherein said at least one cytokine is alpha interferon and wherein said at least one receptor is CD4 receptor.

13. The combination pharmaceutical composition of claim 1, wherein said at least one cytokine is tumor necrosis factor alpha and wherein said at least one receptor is CD4 receptor.

14. A method of treating infectious disease, said method comprising administering to a patient in need thereof, substantially at the same time a) an activated-potentiated form of an antibody to at least one cytokine and b) an activated-potentiated form of an antibody to at least one receptor.

15. The method of claim 14, wherein said activated-potentiated forms of antibodies are administered in the form of a combination pharmaceutical composition.

16. The method of claim 14, wherein said infectious disease is viral infectious disease.

17. The method of claim 16, wherein said viral infectious disease is a disease or condition caused by HIV or associated with HIV.

18. The method of claim 17, wherein said disease and condition caused by HIV or associated with HIV is AIDS.

19. The method of claim 16, wherein said viral infectious disease is viral hepatitis.

20. The method of claim 19, wherein said viral hepatitis is chronic hepatitis C.

21. The method of claim 16, wherein said viral infectious disease is influenza.

22. The method of claim 16, wherein said viral infectious disease is acute respiratory tract infection.

23. The method of claim 14, wherein said infectious disease is bacterial infectious disease.

24. The method of claim 14, 15, 16, 17, 18, 19, 20, 21, 22 or 23, wherein said patient is administered the pharmaceutical composition of claim 9.

25. The method of claim 14, 15, 16, 17, 18, 19, 20, 21, 22 or 23, wherein said patient is administered the pharmaceutical composition of claim 10.

26. The method of claim 14, 15, 16, 17, 18, 19, 20, 21, 22 or 23, wherein said patient is administered the pharmaceutical composition of claim 11.

27. The method of claim 14, 15, 16, 17, 18, 19, 20, 21, 22 or 23, wherein said patient is administered the pharmaceutical composition of claim 12.

28. The method of claim 14, 15, 16, 17, 18, 19, 20, 21, 22 or 23, wherein said patient is administered the pharmaceutical composition of claim 13.

29. The method of claim 15, wherein the combination pharmaceutical composition is administered in one to three unit dosage forms, each of the dosage form being administered from once daily to six times daily.
